(19) United States
(12) Patent Application Publication
(10) Pub. No.: US 2026/0218288 A1
(43) Pub. Date: Jul. 30, 2026

(54) Methods And Targets Of DNA Methylation Entropy

(71) Applicant: The Johns Hopkins University, Baltimore, MD (US)

(72) Inventors: Andrew P. Feinberg, Baltimore, MD (US); Hongkai Ji, Baltimore, MD (US); Yuqi Fang, Baltimore, MD (US); Zhicheng Ji, Baltimore, MD (US); Weiqiang Zhou, Baltimore, MD (US)

(21) Appl. No.: 19/141,970

(22) PCT Filed: Dec. 15, 2023
(86) PCT No.: PCT/US2023/084374

§ 371 Date: Jun. 20, 2025

Related U.S. Application Data
(60) Provisional application No. 63/434,334, filed on Dec. 21, 2022.

(51) Int. Cl. C12Q 1/6869; G16B 20/20; G16B 30/00; G16H 10/40; G16H 10/60; G16H 50/20; G16H 50/30

(52) U.S. Cl. CPC C12Q 1/6869; G16B 20/20; G16B 30/00; G16H 10/40; G16H 10/60; G16H 50/20; G16H 50/30; C12Q 2600/112; C12Q 2600/118; C12Q 2600/154

(57) ABSTRACT

The present disclosure is based on the premise that epigenetic information defines tissue identity and is largely inherited in development through DNA methylation. While studied mostly for mean differences, methylation also encodes stochastic change, defined as entropy in information theory. Analyzing allele-specific methylation in forty-nine human tissue sample datasets, it was found that methylation entropy is associated with specific DNA binding motifs, regulatory DNA, and CpG density. Then applying information theory to 42 mouse embryo methylation datasets, it was found that the contribution of methylation entropy to time- and tissue-specific patterns of development is comparable to the contribution of methylation mean, and methylation entropy is associated with sequence and chromatin features conserved with human. Moreover, methylation entropy is directly related to gene expression variability in development, suggesting a role for epigenetic entropy in developmental plasticity.

### CROSS-REFERENCE TO RELATED APPLICATIONS

This application claims benefit of priority under 35 U.S.C. § 119(e) of U.S. Provisional Application No. 63/434,334, filed Dec. 21, 2022. The disclosure of the prior application is considered part of and is herein incorporated by reference in the disclosure of this application in its entirety.

### STATEMENT REGARDING GOVERNMENT FUNDING

This invention was made with government support under grant 1933303, awarded by the National Science Foundation, and grants DK119129, HG010889, and HG009518, awarded by the National Institutes of Health. The government has certain rights in the invention.

### BACKGROUND OF THE INVENTION

### Field of the Invention

The present invention relates generally to DNA methylation and more specifically to epigenetic entropy and methods for detecting a developmental defect or cancer in a subject.

### Background Information

Epigenetic information defines tissue identity and is largely inherited in development through DNA methylation. While studied mostly for mean differences, methylation also encodes stochastic change, defined as entropy in information theory. Analyzing allele-specific methylation in 49 human tissue sample datasets, it was found that methylation entropy is associated with specific DNA binding motifs, regulatory DNA, and CpG density. Then applying information theory to 42 mouse embryo methylation datasets, it was found that the contribution of methylation entropy to time- and tissue-specific patterns of development is comparable to the contribution of methylation mean, and methylation entropy is associated with sequence and chromatin features conserved with human. Moreover, methylation entropy is directly related to gene expression variability in development, suggesting a role for epigenetic entropy in developmental plasticity.

DNA methylation, a covalent modification of the nucleotide cytosine, heritable during cell division at CpG dinucleotides, is a key component of the epigenetic information, i.e., independent of the DNA sequence itself, defining cell type identity and developmental state. Differences in DNA methylation levels between individuals can be driven by nearby DNA sequence differences termed methylation quantitative trait loci (mQTLs), and DNA methylation level is generally inversely related to mean gene expression levels, particularly at gene promoters. In addition to DNA methylation levels, DNA methylation stochasticity, more formally defined as entropy in information theory, is related to processes involving plasticity, such as the epithelial-mesenchymal transition in cancer and differentiation potency and could also help to regulate developmental plasticity. However, it remains poorly understood how epigenetic entropy is influenced by DNA sequence, how entropy might function, its relationship to developmental state or transcription factor binding sites, or what might be its effect on gene expression.

Information theory is the quantitative field that measures and analyzes information and entropy in its transmission, and it is thus a natural fit to address stochasticity in methylation. FIG. 1A illustrates this idea applied to multiple consecutive CpG sites on the same DNA molecule. The two examples have similar mean methylation levels but markedly different entropy. The example on the right shows substantial variation from molecule to molecule in terms of the combinatorial configurations of the binary methylation states of multiple CpG sites in the same molecule, and hence high entropy, whereas the example on the left has little such variation and low entropy. Our overall goal was to relate differences in entropy (compared to mean) to the methylation potential energy landscape, genetic sequence, regulatory DNA, transcription factor binding, embryonic development, and gene expression (FIG. 1A).

Entropy analysis has been applied to whole-genome bisulfite sequencing methylation data, but entropy analysis has not incorporated the role of the underlying DNA sequence itself in its control or been applied to embryonic development. An information theory method was first applied to measure DNA methylation mean and entropy that can distinguish the two alleles of a gene, providing the perfect control since the two alleles are present in exactly the same cells. Data from 49 polymorphic human samples were analyzed obtained from the Roadmap Epigenomics Project, which contain not only DNA methylation sequencing data, but also complete DNA sequence (SNPs). Three questions were asked comparing entropy and mean methylation (outlined in FIG. 1B): (1) How are they related to transcription factor binding site?(2) How are they related to CpG density? and (3) How are they related to regulatory DNA?

In order to better understand the functional role of methylation entropy, an information-theoretic analysis of mouse embryonic development, examining comprehensive whole-genome DNA methylation data from the ENCODE3 project on 7 individual tissue types during mouse development at 6 developmental time points was then performed, addressing five further questions (outlined in FIG. 1B): (1) Does this information theory-based approach identify tissue- and time-dependent changes in the methylation landscape?(2) What is the relative contribution of mean methylation and methylation entropy to these developmental landscape changes?(3) Can we integrate these results with orthogonal measures of transcription factor binding sites, CpG density, and regulatory DNA in order to understand the role of DNA methylation entropy?(4) Are these features conserved between mouse and human? and (5) What is the functional relationship between methylation entropy and gene expression variability, usingsingle-cell RNA-seq data from both mouse and human?

### SUMMARY OF THE INVENTION

The present invention is based on the seminal discovery that there are striking associations of methylation entropy with genomic and developmental processes, the sequences that may drive them, and their relationship to developmental gene expression.

In one embodiment, the invention provides a method of detecting a developmental defect in a subject comprising identifying changes in a normalized methylation entropy (NME) level in a sample from the subject, thereby detecting a developmental defect in the subject.

In another embodiment, the invention provides method of detecting cancer, cancer recurrence and/or cancer recurrence risk in a subject comprising identifying changes in a normalized methylation entropy (NME) level in a sample from the subject, thereby detecting cancer, cancer recurrence and/or cancer recurrence risk in the subject. In one aspect, changes in the normalized methylation entropy (NME) level are determined for each allele.

In one aspect of the invention, identifying changes in an NME level comprises identifying a change in DNA methylation landscape in the sample from the subject. In one aspect, the identifying a change in methylation landscape comprises calculating an information-theoretic distance between a methylation potential energy landscape quantified by an uncertainty coefficient (UC) in the sample from the subject and a methylation potential energy landscape quantified by an UC in a reference sample. In one aspect, the UC measures an inherent information in a sample corrected for entropy.

The invention also envisions deriving tissue-specific developmental trajectories from the information-theoretic content of DNA methylation, wherein identifying regions with high UC in the sample from the subject as compared to the UC in the reference sample indicates a developmental change in the subject.

For example, the developmental change is a tissue-specific developmental change or a time-dependent developmental change. In one aspect, the tissue-specific developmental change comprises changes in embryonic facial prominence, liver, heart, limb, forebrain, midbrain and/or hindbrain developmental change. In one aspect, the developmental change comprises epithelial to mesenchymal transition. For example, deriving tissue-specific developmental trajectories comprises identifying entropy-associated motifs undergoing methylation landscape changes. In one aspect, the entropy-associated motifs are selected from the motifs identified in Table 2.

### BRIEF DESCRIPTION OF THE DRAWINGS

FIGS. 1A-1B illustrate the Study overview. FIG. 1A is a conceptual illustration of applying information theory to a set of DNA reads from WGBS. Each row is a single DNA molecule representing one read, and the dots on the line represents the CpG location. The blue dots correspond to unmethylated CpG and the red dots correspond to methylated CpG. The mean methylation level (MML) on the left panel is the same as the MML on the right panel. However, when considering the combinatorial configuration of methylation states of multiple CpGs in the same molecule, the left panel shows small variation among reads (rows), whereas the right panel shows large variation. As a result, the right panel has a much larger normalized entropy (NME) than the left panel (see Methods and Materials). FIG. 1B is a table illustrating a summary of the analyses performed in this study, which examines methylation entropy and its relationship to functional genomic features including transcription factor binding sites, CpG density and regulatory DNA; and relationship to functional consequences including the embryonic development, contribution of methylation entropy and mean methylation to methylation landscape, orthogonal relationships to transcription factor binding sites, CpG density and regulatory DNA, conservation of those features between human and mouse, and the association between gene expression variability and methylation entropy.

FIG. 2 illustrates examples of allele-specific methylation mean and entropy analysis. Four example regions with large methylation mean difference (top panel) or large methylation entropy difference (bottom panel) between two alleles. For each region, the continuous horizontal lines represent individual sequencing reads. The vertical lines are the location of the SNPs used to distinguish two alleles. The dark dots represent methylated CpG sites, and the light dots represent unmethylated CpG sites. The top panel shows two example regions located at two known imprinted genes, PLAG1 and KCNQ1. Consistent with the known association between imprinting and promoter methylation, in one allele almost all CpG sites are methylated and in the other allele almost all CpG sites are unmethylated. The lower panel shows two genes, ASPG and TMC4, with high methylation entropy differences inside the gene. For ASPG, the allele with genotype A has low entropy (0.13) as indicated by most of the reads having the same methylation status. The allele with the genotype of G has high entropy (0.79) as indicated by the highly stochastic color pattern. For TMC4, the allele with the genotype of C has low entropy (0.15), while the allele with the genotype of T has high methylation entropy (0.86) as indicated by the highly variable methylation patterns across reads.

FIGS. 3A-3C illustrate allelic sequence relationship to NME. FIG. 3A shows the analysis for each SNP genotype in all 52 possible trinucleotide contexts. Log(Odds ratio) (x-axis) greater than 0 means the right trinucleotide is more likely to have higher entropy than the left trinucleotide than random expectation. If the SNP changes the number of CpG sites, the allele with fewer CpG sites is shown on the right. For each SNP genotype, the trinucleotide contexts associated with the largest allelic entropy differences (i.e., highest odds ratio) were often the ones that change the CpG number. Error bar is used to show the upper and lower 95% of CI of the log(OR). FIG. 3B shows the distribution of NME comparing the alleles with higher or lower numbers of CpG. For the regions that have CpG number differences, the allele is labeled as “more CpG” for the allele that contains more CG than the allele labeled “fewer CpG”. The allele with more CG has significantly lower NME than the allele with fewer CpG (paired one-sided t-test, P<2.2×10⁻¹⁶). The distribution of NME for all alleles is displayed as a reference. C. NME and MML, regardless of SNPs, of genomic regions surrounding transcription start sites (20 kb around each TSS) stratified based on CpG density. The CpG density was defined as the ratio of the observed CpG number to the expected CpG number in each region (see Example 1). The centerline represents the median and the upper and lower lines are the first and third quantile. As CpG density increases, the NME decreases. MML showed an abrupt decrease at high CpG density while NME had a gradual decrease as CpG increased.

FIGS. 4A-4F illustrate the information-theoretic analysis of methylation landscape in mouse embryonic development. FIG. 4A shows a multi-dimensional scaling (MDS) plot of mouse embryonic samples from different tissues and developmental time points based on samples' pairwise distance using uncertainty coefficient (UC). Samples (dots) are coded either by tissues (top) or by developmental time points (bottom). Samples from the same tissue or similar tissues tended to be clustered together. The MDS also captured samples' temporal ordering, with samples from earlier developmental time points being closer to a common center (labeled by the red dot in the bottom plot) and samples from later time points gradually moving away from the center toward different directions representing different tissues. FIG. 4B shows a Heatmap of UC values in mouse embryogenesis revealed tissue- and developmental stage-specific DNA methylation landscape changes. Regions with high UC in only one tissue were grouped into 10 clusters, which were ordered according to the consecutive time points with the highest UC. The clustering reflected the temporal cascade of methylation landscape changes in that tissue. High UC means large methylation change (including mean and entropy changes) and low UC means small change. The temporal patterns were tissue-specific, as patterns observed in one tissue cannot be observed in other tissues. FIG. 4C shows a gene ontology (GO) analysis demonstrating the biological significance of UC-based developmental clustering. For each tissue and region cluster identified in FIG. 4B, enriched biological functions associated with enhancer regions in the cluster are identified and the top 5 GO terms with largest fold-enrichment are shown (rows: GO terms; columns: tissues and region clusters; “*”: FDR<=0.1). The enriched GO terms revealed tissue-specific functions. (Note: although GO terms in cluster 6, hindbrain have high fold-enrichments in other tissues, they are not significant.) The GO terms in the heart followed the development order, i.e., atrial septum and cardiac atrium development were observed in early stages and heart valve development in late stages. Similarly, in the forebrain, the GO terms related to neuron development were observed in early stages and brain subdivision formation in later stages. FIG. 4D is a bar plot showing the relative contribution of MML and NME to the methylation landscape changes as characterized by UC. In each tissue, regions were classified into four categories based on whether a region's UC change across time points was predominantly correlated with the change in MML, NME, both, or uncertain. The proportion of each category among all regions that have high UC in at least one pair of time points is shown. In all tissues, there were more regions where UC is predominantly correlated with NME change than the regions where UC is predominantly correlated with MML change, suggesting a larger role of methylation entropy than mean methylation in shaping the dynamic changes of methylation landscape. FIG. 4E illustrates a gene ontology (GO) analysis demonstrating the biological significance of regions whose UC is predominantly correlated with NME using the same method as in FIG. 4C. All tissues show tissue-specific GO terms from those regions whose UC is predominantly correlated with NME. FIG. 4F illustrates a gene ontology (GO) analysis demonstrating the biological significance of regions whose UC is predominantly correlated with MML. Some tissues show tissue-specific GO terms such as EFP, forebrain, limb and midbrain.

FIGS. 5A-5G illustrate the association of methylation entropy changes with transcription factor binding motifs. Scatterplots comparing the enrichment level of each TF motif in regions where DNA methylation changes were predominantly correlated with NME change versus the enrichment level of the same motif in regions where methylation changes were predominantly correlated with MML change. FIG. 5A shows the results of the analysis run for embryonic facial prominence (EFP), in the mouse embryonic development dataset. FIG. 5B shows the results of the analysis run for forebrain, in the mouse embryonic development dataset. FIG. 5C shows the results of the analysis run for heart, in the mouse embryonic development dataset. FIG. 5D shows the results of the analysis run for hindbrain, in the mouse embryonic development dataset. FIG. 5E shows the results of the analysis run for limb, in the mouse embryonic development dataset. FIG. 5F shows the results of the analysis run for liver, in the mouse embryonic development dataset. FIG. 5G shows the results of the analysis run for midbrain, in the mouse embryonic development dataset. For each tissue, motifs that were more enriched in the regions where UC was predominantly correlated with NME change compared to regions where UC was predominantly correlated with MML change (entropy-associated motifs, marked with blue color) as well as motifs that were more enriched in regions where UC was predominantly correlated with MML change than the regions where UC was predominantly correlated with NME change were identified (mean-associated motifs). Motifs that were also identified in the human analysis which prefer high NME are marked with text labels. Entropy-associated motifs that prefer high NME in the human analysis are also marked. Mean-associated motifs that preferred high NME in the human analysis are marked. Among the entropy-associated motifs, a substantial number (32, 27, 16, 24, 26, 27, and 19 motifs from heart, forebrain, limb, EFP, midbrain, hindbrain, and liver, respectively) also prefer high NME in the human analysis such as KLF4 and KLF5.

FIGS. 6A-6B illustrates the association of methylation entropy changes with gene expression variability. FIG. 6A shows the association between NME surrounding transcription start sites (TSS) and gene expression variability. An example showing NME near TSS was lower for the gene with low mean-adjusted expression variability (MAV) than the gene with higher MAV. Genes were stratified based on the quartiles of their MAV. For each stratum, the average NME surrounding TSS across all genes in the stratum is shown. The genes in the lowest MAV quantile had the lowest NME near their TSS. The genes in the highest MAV quantile had much higher NME near their TSS. The Pearson correlation between genes' MAV and NME was 0.269 (P<2.2×10⁻¹⁶). FIG. 6B is a heatmap showing NME values within +−500 bp of genes' TSS in different samples. Each row is a sample. In each sample, genes were stratified based on their expression variability using quantiles of MAV (X-axis), and the heatmap shows the average NME of all genes in each stratum. Increasing NME was observed as the MAV quantile increases except for embryonic cell lines, indicating that gene expression variability (MAV) is correlated with NME in regions surrounding genes' TSS.

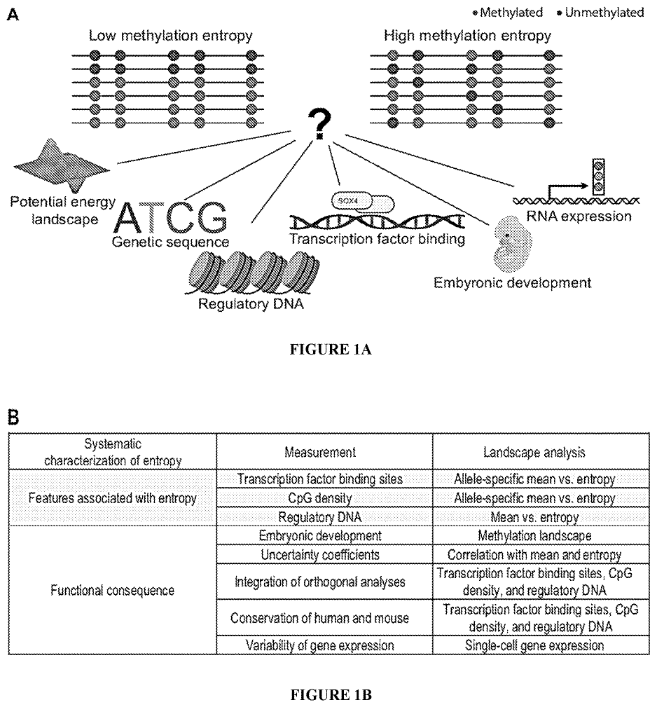

### DETAILED DESCRIPTION OF THE INVENTION

The present invention is based on the seminal discovery that there are striking associations of methylation entropy with genomic and developmental processes, the sequences that may drive them, and their relationship to developmental gene expression.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the invention provides a method of detecting a developmental defect in a subject comprising identifying changes in a normalized methylation entropy (NME) level in a sample from the subject, thereby detecting a developmental defect in the subject.

As used herein, the term “developmental defect” is meant to include any congenital anomalies occurring in any organ. The developmental defect can result from intrinsic causes including genetic defects (mutations), endogenous chromosomal imbalances (e.g., meiotic nondisjunctions), endogenous metabolism (e.g., phenylketonurea), and perhaps failures in the complex developmental processes themselves; or extrinsic causes including the enormous variety of environmental inputs such as infection, nutritional deficiencies and excesses, life-style factors (e.g., alcohol), and closer to the concerns of this committee, the myriad agents-pharmaceuticals, synthetic chemicals, solvents, pesticides, fungicides, herbicides, cosmetics, food additives, natural plant and animal toxins and products, and other environmental chemicals-encountered by humans.

In another embodiment, the invention provides method of detecting cancer, cancer recurrence and/or cancer recurrence risk in a subject comprising identifying changes in a normalized methylation entropy (NME) level in a sample from the subject, thereby detecting cancer, cancer recurrence and/or cancer recurrence risk in the subject. In one aspect, the normalized methylation entropy (NME) levels are determined for each allele.

Cancer is a group of diseases involving abnormal cell growth with the potential to invade or spread to other parts of the body. In 2015, about 90.5 million people had cancer, about 14.1 million new cases occur a year and it caused about 8.8 million deaths (15.7% of deaths). The most common types of cancer in males are lung cancer, prostate cancer, colorectal cancer and stomach cancer. In females, the most common types are breast cancer, colorectal cancer, lung cancer and cervical cancer.

The term “cancer” refers to a group of diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS—Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland'Cancer, Childhood; Sarcoma, Ewing's Family of Tumors; Sarcoma, Kaposi's; Sarcoma (OsteosarcomaVMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

As used herein, the term “cancer recurrence” is meant to refer to both local recurrence and distant recurrence or metastases, when a primary tumor if found at a secondary location, that is different from the primary location.

In one aspect of the invention, identifying changes in an NME level comprises identifying a change in DNA methylation landscape in the sample from the subject. In one aspect, the identifying a change in methylation landscape comprises calculating an information-theoretic distance between a methylation potential energy landscape quantified by an uncertainty coefficient (UC) in the sample from the subject and a methylation potential energy landscape quantified by an UC in a reference sample. In one aspect, the UC measures an inherent information in a sample corrected for entropy.

The invention also envisions deriving tissue-specific developmental trajectories from the information-theoretic content of DNA methylation, wherein identifying regions with high UC in the sample from the subject as compared to the UC in the reference sample indicates a developmental change in the subject.

For example, the developmental change is a tissue-specific developmental change or a time-dependent developmental change. In one aspect, the tissue-specific developmental change comprises changes in embryonic facial prominence, liver, heart, limb, forebrain, midbrain and/or hindbrain developmental change. In one aspect, the developmental change comprises epithelial to mesenchymal transition. For example, deriving tissue-specific developmental trajectories comprises identifying entropy-associated motifs undergoing methylation landscape changes. In one aspect, the entropy-associated motifs are selected from the motifs identified in Table 2.

DNA methylation analysis can be performed by methods known to those of skill in the art. By way of example and not limitation, Table 3 provides possible methods that could be applied in the methods of the invention.

Presented below are examples contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Materials and Methods

### SNP Data Processing

The revealing Single Nucleotide Polymorphisms (SNPs) information for subjects H9, HUES64, STL001, STL002, STL003, skin03, HuFGM02, 149,150, and 112 were extracted from the Roadmap Epigenomics database. Whole-genome sequencing data (WGS) for the H1 cell line was downloaded from PRJNA285681 (SRR2048232). The WGS reads were trimmed using Trim Galore (v0.5.0). The trimmed reads were aligned to the hg19 reference genome using the Arioc WGS alignment package(v1.40). Duplicated reads from polymerase chain reaction (PCR) products were removed using Picard tools (MarkDuplicates v2.18.13). SNPs were called using GATK HaplotypeCaller (v4.0.0) and dbSNP build 15.

### Allele-Specific Methylation Detection.

The Whole-genome bisulfite sequencing (WGBS) data was aligned, and reads were assigned to each allele in the same way as the correlated potential energy landscape (CPEL) pipeline. The first and last 5 bp of each read were not used in the analysis. To perform haplotype-dependent allele-specific methylation analysis, the Julia package CpelAsm.jl, a recently developed method was used. For a given haplotype, CpelAsm estimates an allele-specific epigenetic landscape of the random methylation state X ∈ λ={0,1}^{N}, where N is the number of CpG sites in the region, by performing maximum likelihood estimation on a set of M independent WGBS reads obtained for each allele as described in the CpelAsm, resulting in a probability mass function of the methylation state p(x), x ∈ λ, for each allele (allele subscript not shown for notational simplicity hence forth). Once the parameters of the allele-specific models are estimated, CPEL computes the allele-specific mean methylation level (MML) μ as well as the normalized methylation entropy (NME) h, a measure of epigenetic stochasticity, for each allele. In particular, the MML of a given allele is given by

$$μ = \frac{1}{N} {\sum }_{n = 1}^{N} E [ {X}_{n} ]$$

where Xₙ is the random methylation state of the n-th CpG site, and E[Xₙ] denotes the expected methylation level of the n-th CpG site. The corresponding NME is given by

$$h = - \frac{1}{N} {\sum }_{x ∈ 𝒳} p ( x ) {log}_{2} p ( x )$$

Both quantities are normalized to produce values in the range [0,1]. Then, CPEL computes the absolute MML difference and NME difference between two alleles. The first statistic measures the absolute difference in MML (dMML) between haplotype alleles, and it is simply given by |μ₁−μ₂|. The second statistic measures the absolute difference in NME (dNME) between haplotype alleles, and it is given by |h₁−h₂|. Thus, a large NME difference suggests that the methylation state in one allele is highly stochastic while that of the other allele behaves almost deterministically. In order to determine whether the allelic difference in MML or NME is statistically significant, null statistics were generated from homozygous regions of the genome to obtain an empirical null distribution for each test statistic (dMML, dNME). However, the distribution of the null statistics will be a function of the number of CpG sites N. Therefore, a set of null statistics was generated for each N considered. To generate a null statistic, a genomic window with no SNPs was randomly chosen and a contiguous set of N CpG sites was selected. Next, the reads mapping to that region were randomly split into two groups, which simulate two alleles. A CPEL model is estimated for each simulated allele, and the corresponding statistics are computed to produce a null statistic. This process is repeated until 1,000 null statistics for each N were obtained. These sets of null statistics are used then to compute a p-value for each test statistic in each heterozygous region. In the case of the SNP being located at C or G in the CpG that changes CpG number, the SNP-containing CpG is not included in the CPEL model in both alleles. This ensures a fair comparison between the two alleles. Subsequently, CpelAsm used the Benjamini-Hochberg procedure to compute adjusted p-values to control the false discovery rate (FDR) in the statistical output. The FDR≤0.1 was used to determine the significance of the MML difference and NME difference.

Regions with Large MML Difference Enrichment Analysis

The list of imprinted genes was downloaded from the Geneimprint website. The contingency table was constructed by counting the occurrence that the regions showing large MML differences in the promoter of the imprinted gene versus non-imprinted gene. The odds ratio was calculated, and the two-sided Fisher's exact test was performed using the contingency table where rows are regions with or without significant dMML and columns are regions at the promoter region of imprinted or non-imprinted genes. The same enrichment analysis was done for the mono-allelic expressed gene (MAE) where rows are regions with or without significant dMML and columns are regions at the promoter region of MAE genes or non-MAE genes.

### Human Motif Binding Analysis

The motifBreakR package in the default setting was used to first map the motif obtained from the JASPAR database at each SNP to predict the transcription factor binding sites (TFBSs). For each motif site, the binding probability of the corresponding motif in each allele by considering the polymorphism was then evaluated. For each motif site, the occurrence of the region with higher binding probability in the allele with higher NME or MML than the other allele was counted. This observation should be 50% by random chance. On the contrary, if the occurrence of such regions is significantly higher than 50%, using the binomial test, it suggests that a higher transcription factor (TF) binding probability is associated with higher NME or MML. Similarly, the analysis was performed to identify motifs that are associated with lower NME or MML.

### Analyzing the Association Between NME and SNP

The trinucleotide context near the SNP was examined, i.e., 1 bp before and after the SNP. For each type of SNP, there are 16 possible trinucleotide changes. Trinucleotide changes that are reverse complements were merged, e.g., GCG→GTG and CGC→CAC. The trinucleotide was connected by → in the direction of SNP. The direction of SNP was determined by decreasing CG if there are CG changes in the trinucleotide, e.g., GCG→GTG. Allele1 is the allele on the left, e.g., GCG, and the allele2 is the allele on the right, e.g., GTG. The NME difference was calculated for each region using NME in the allele1 minus the NME in allele2. Note that for both alleles the NME was calculated without using the SNP-containing CpG to ensure a fair comparison between alleles. For each trinucleotide context, the contingency table was constructed by counting the occurrence that the NME in allele1 is smaller than the NME in allele2 in that trinucleotide context versus not in that trinucleotide context. The log (Odds ratio) and p-value were calculated using the two-sided Fisher's exact test. The Benjamini-Hochberg correction was used to calculate the FDR for significance.

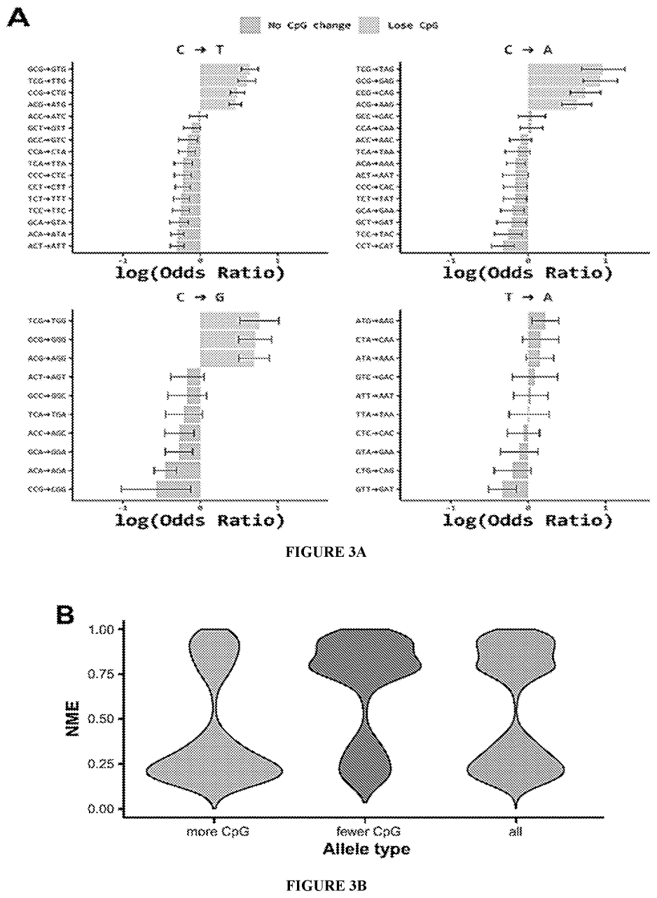

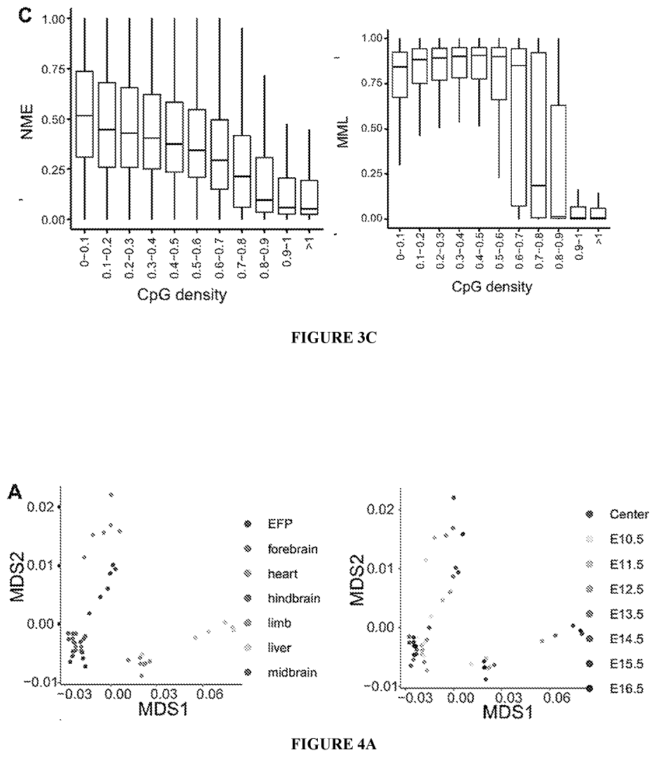

Some regions contain more than 1 SNPs that change the CpG number. To calculate the effect of the CpG number in the allele on NME, the CpG number changes and NME changes between two alleles were calculated. The analyzed regions with different CpG numbers between two alleles due to SNP and significant NME difference (FDR≤0.1) were selected. Among those regions, the distribution of NME in the allele with more CpGs was plotted in FIG. 3B and the distribution of NME in the allele with fewer CpG was plotted in FIG. 3B. The one-sided t-test was used to test the significance NME difference between two alleles because it was found the allele with more CpG tends to have smaller NME when analyzing SNPs. The NME in both alleles in all the regions with significant NME differences between the two alleles, regardless of CpG number, was plotted in FIG. 3B as a reference.

The number of CpG was counted in each allele with an extended region (500 bp). The expected number of CpG was calculated using equations from

$$\frac{{( ( {N}_{c} + {N}_{G} ) / 2 )}^{2}}{size of the region}$$

The density was calculated for each allele by the number of CpG/expected CpG. The CpG density difference was calculated by the difference of CpG density between two alleles. The regions showing significant NME difference (FDR≤0.1) were used to calculate the correlation between CpG density difference and NME difference.

Human NME and MML Calculation without Separating Alleles

Allele-specific analysis can only be performed for regions with heterozygote SNPs. To analyze the whole genome regardless of SNPs, the WGBS data for human samples were aligned without assigning the allele. The first and last 5 bp of each read were not used. The target region was divided into 250 bp segments. For each segment, regardless of the allele of origin, the MML and the NME were calculated as described in the ‘Allele-specific methylation detection’ section. The CpG density-dependent nature of differential NME (dNME) suggested that simply lumping data from both alleles might give a false impression of information-theoretic entropy. If one simply pooled the reads from the two alleles with a 50-50 mixture of a highly methylated and lowly methylated allele of a gene, both with near-zero entropy, combined reads would sometimes falsely appear to be relatively high entropy. That is exactly what would happen when examining imprinted regions, where one allele is in fact methylated depending on the parent of origin. Indeed, if one computes NME without regard to an SNP, there is a false skewing to high NME in regions with significant MML difference such as genes with imprinting; while that is not the case for regions without significant MML difference. Since regions with allelic mean methylation imbalances were rare across the genome (0.422% of all regions analyzed), and since the main difference between the mean allelic methylation entropy and methylation entropy regardless of SNP were in such regions, regions showing mean methylation imbalances from this analysis were excluded. As a result, in this whole-genome analysis, NME is expected to be similar to the mean allelic NME of the two alleles at each locus.

### NME and CpG Density Analysis

NME and MML were calculated within 20 kb of transcription start site (TSS) as described in the ‘Human NME and MML calculation without separating alleles’ section. T\hat{ }he CpG density was computed for each region using (total number of CpG)/(expected number of CpG). The expected number of CpG was calculated using the same way used in ‘Analyzing the association between NME and SNP’. The correlation between CpG density and NME or MML regardless of the allele was calculated using the two-sided Pearson correlation test.

### Human Motif Analysis

The non-redundant CORE motifs were downloaded from JASPAR. The 630 human motifs representing 586 TFs or TF complexes were then mapped to the human genome using CisGenome. For each motif, the mapped motif sites were grouped into two classes (regulatory and non-regulatory) based on 167 ENCODE DNase-seq samples. Regulatory DNA is defined as the union set of chromatin accessible sites across the 167 DNase-seq samples which were downloaded from github.com/WeiqiangZhou/BIRD-data. Briefly, to obtain the regulatory regions in human, the aligned DNase-seq data (alignment based on hg19) from 167 ENCODE samples (representing 74 cell types) were downloaded from encodeproject.org/. Genomic regions from chromosome Y were excluded. Then, the genome was divided into 250 base pair (bp) non-overlapping bins. The number of reads mapped to each bin was counted for each DNase-seq sample. To adjust for different sequencing depths, bin read counts for each sample were first divided by the sample's total read count and then scaled by multiplying a constant (minimum total read count from all the samples). Since most genomic loci are noise rather than regulatory elements, genomic loci were filtered to exclude those without strong DNase I hypersensitivity (DH) signal in any training DNase-seq sample. The filtering was done in three steps. First, genomic bins with normalized read count ≤20 in all samples were excluded. Second, bins with normalized read count larger than 10,000 in ≥1 sample were considered abnormal and therefore also excluded. Third, a signal-to-noise ratio (SNR) was computed for each bin in each sample, and bins with SNR≤3 in all samples were considered as noise and filtered out. To compute SNR of a genomic bin in a sample, 40 bins were first collected in the neighborhood of the bin in question. The average DH level of these bins was computed to serve as the background. The log 2(SNR) was defined as log 2([DH level of a bin]/[background]). To obtain the regulatory regions in mouse, the DNase-seq peak files (mm10) from 72 samples which are from the similar tissues and developmental stages as the DNA methylation data was downloaded. Similarly, genomic regions from chromosome Y were excluded. The peak regions were merged from all the samples and divided into 250 bp bins. To get a set of non-regulatory DNA, CisGenome was used to obtain genomic regions that are not located in the regulatory DNA but have a similar distribution of distance to TSS with the regulatory DNA. For each motif, motif sites that overlap with regulatory DNA were labeled regulatory motif sites, and motif sites that overlap with non-regulatory DNA were labeled as non-regulatory motif sites. For this, methylation entropy was used to characterize the NME of each motif site in regulatory DNA and non-regulatory DNA. The NME regardless of alleles was calculated using the method described in the ‘Human NME and MML calculation without separating alleles’ section. For each TF, the median value of NME across all motif sites in each of the two motif site classes (i.e., regulatory and non-regulatory) was computed. Based on the median NME for each TF across different samples, a two-sided Wilcoxon signed-rank test was then used to test whether NME is different between the two classes of sites. To adjust for multiple testing, the false discovery rate (FDR) was calculated using Benjamini-Hochberg procedure. Similarly, the MML between motif sites in regulatory DNA and non-regulatory DNA were compared for each TF. A two-sided Wilcoxon signed-rank test was also applied to test whether MML is different between the two classes of sites.

### Mouse Embryonic Development Data Analysis

The aligned WGBS bam files were manually downloaded from the ENCODE3 database. The bam files were sorted using samtools v1.9 and deduplicated using Picard tools MarkDuplicates (v2.23.3-4) function. Uncertainty coefficient (UC) is the Jensen-Shannon distance (JSD) normalized by entropy between the DNA methylation landscapes of two time-points which captures both mean methylation changes and methylation entropy differences. The difference in methylation landscapes between the two samples s₁ and s₂ was quantified by computing the uncertainty coefficient (UC)

$$UC ( X ; S ) = \frac{1}{N} \frac{I ( X ; S )}{h ( X )}$$

where I(X;S) is the mutual information between the methylation state X and the sample S, and it is given by

$$\begin{matrix}I ( X ; S ) = - {\sum }_{x} {\sum }_{s ∈ \{ s 1 , s 2 \}} p ( X = x , S = s ) {log}_{2} \frac{p ( X = x , S = s )}{p ( X = x ) p ( S = s )} \\ = - {\sum }_{x} {\sum }_{s ∈ \{ s 1 , S 2 \}} p ( X = x , S = s ) {log}_{2} \frac{p ( X = x \vert S = s )}{p ( X = x )}\end{matrix}$$

and h(X) is the NME under a model where the sample S has been integrated out. It has been shown that I(X; S)=D²(p₁, p₂) where D(p₁, p₂) is the Jensen Shannon Divergence (JSD) that measures the similarity between two probability distributions p₁=p(X=x|S=s₁) and p₂=p(X=x|S=s₂). Large JSD means a large difference in methylation probability distribution between two samples. Given the linear dependency of UC(X;S) on the mutual information I(X;S), large values of UC(X;S) imply a large difference between samples. The NME, MML, differential NME, differential MML, and UC were then calculated using the CPEL pipeline for each genomic region. Regions that have lower than 10× coverage were excluded.

For each tissue type, regions with UC>0.1 were first analyzed using unsupervised clustering. Because more than 28% of them were found to only have UC>0.1 in one tissue, the region with UC>0.1 only within that tissue (not in other tissues) was selected for at least one pair of time points. UC for all consecutive pairs of time points (e.g., E10.5 and E11.5, E11.5 and E12.5) were standardized to have mean 0 and variance 1 for each region across consecutive pairs of time points. The standardized values were then used to computationally group regions into 10 UC clusters with k-means clustering. The k-means clustering was performed 10 times with different random seeds. The regions that were always clustered in the same clusters were defined as core-cluster regions. The rest of the regions were assigned to each core cluster based on their correlation to the mean UC of the core cluster. The 10 UC clusters were ordered according to which consecutive time points have the largest UC.

UC>0.1 was selected as the cutoff because the top 5% of all UC values is around 0.1. the other UC cutoffs further tested included, 0.025 (~top 30%), 0.05 (~top 15%), 0.15 (~top 3%), 0.2 (~top 1%), using the same method.

### Mouse Gene Ontology (GO) Analysis

Mouse GO analysis was done for each cluster in each tissue. For regions overlapping with enhancers, the target genes were annotated according to the published dataset. The genes for which complete methylation data were available in all samples at enhancer regions were used as the background gene list for GO analysis. For each cluster, the regions overlapping with enhancers were annotated to the corresponding gene and were used as target genes for GO analysis. The R package topGO was used to perform GO analysis using the classicFisher method. The GO terms with more than 10 annotated genes were retained. For a given GO term, let N denote the number of genes with an enhancer that has methylation landscape change, and let M denote the number of other genes in this GO term. Let Q denote the number of genes not in this GO term but with an enhancer that has methylation landscape change, and let R denote the number of other genes not in this GO term. The association between the GO term and the methylation landscape change is tested by examining N. The GO terms with N=0 were excluded. The P-values were calculated conditional on the knowledge that the GO terms with N=0 are excluded. In other words, the probability of observing N=n genes in a GO term whose enhancers have methylation landscape changes conditional on N≥1 is:

$$P ( N = n \vert N \geq 1 ) = \frac{{P}_{hyper} ( n )}{1 - {P}_{hyper} ( 0 )} \\ where {P}_{hyper} ( N ) = \frac{( \begin{matrix}N + M \\ N\end{matrix} ) ( \begin{matrix}R + Q \\ Q\end{matrix} )}{( \begin{matrix}N + M + R + Q \\ Q + N\end{matrix} )}$$

Therefore, the P-value for observed N, i.e., (N_{observed}) is:

$$P - value = {\sum }_{n \geq {N}_{observed}} P ( N = n \vert N \geq 1 )$$

The Benjamini-Hochberg procedure was used to correct p-values for multiple hypothesis testing. The GO terms with fold change (FC) greater than 1.5 were reported. The final GO terms for each cluster were ranked by FDR and FC was used to break ties. The top 5 GO terms were selected from each cluster to generate the heatmap. The GO terms for all tissues and clusters were plotted together. The GO terms that do not have FDR≤0.2 in all tissue-cluster were excluded. The rows in the heatmap were ordered based on which columns show the highest FC.

For regions overlapping promoters, defined as within 2 kb of TSS, the genes were annotated to that TSS and the GO analysis was performed using the same procedure. The genes for which complete data in all samples at promoter regions were available were used as the background list.

The other UC cutoffs were further tested, 0.025 (~top 30%), 0.05 (~top 15%), 0.15 (~top 3%), 0.2 (~top 1%), using the same method.

### Categorize Regions Based on UC-dNME and UC-dMML Correlation

For each tissue, the correlation between NME change and UC (dNME-UC correlation) and the correlation between MML change and UC (dMML-UC correlation) were calculated. The labels of the NME/MML differences were randomly permuted 20 times, recalculated the correlations between permuted NME/MML and UC as the null distribution, and obtained empirical p-values as the tail areas under the null distribution. FDRs were then calculated based on p-values using the Benjamini-Hochberg procedure. Some regions have a high dMML-UC correlation and dNME-UC correlation. To distinguish those regions from the regions that only have high dNME-UC correlation or dMML-UC correlation, different clusters of regions were identified based on the density of dMML-UC correlation and dNME-UC correlation. To that end, the difference between dNME-UC correlation and dMML-UC correlation (dNME-dMML difference) as well as the mean of two correlations for each region was calculated. The mean correlation was binned with a 0.05 interval. For each bin, the first local minimum for the density of dNME-UC and dMML-UC difference below 0 and above 0 was searched for (negative local minimum and positive local minimum). The regions with values below the negative local minimum suggested the dMML-UC correlation is larger than the dNME-UC correlation. Thus, among the regions with significant dMML-UC correlation (FDR≤0.2), the regions with dNME-dMML difference below the negative local minimum or have dMML-UC correlation greater than 0 while dNME-UC correlation smaller than 0, were categorized as regions whose UC is predominantly correlated with MML change (predominantly MML-correlated regions), meaning that their UC changes across time can be mainly explained by the MML changes. On the other hand, the regions with values above positive local minimum suggested the dNME-UC correlation is larger than the dMML-UC correlation. Thus, among the regions with significant dNME-UC correlation (FDR≤0.2), the regions with dNME-dMML difference above the positive local minimum or their dNME-UC correlation is greater than 0 while dMML-UC correlation is smaller than 0, were categorized as regions whose UC is predominantly correlated with NME change (predominantly NME-correlated regions), meaning that their UC changes across time can be mainly explained by the NME changes. The regions that have values between two local minimums and have significant dNME-UC correlation and dMML-UC correlation (FDR≤0.2) were categorized as regions whose UC is correlated with both NME and MML, meaning that both MML and NME changes contribute to the temporal changes of UC (Both). The regions that have insignificant dMML-UC correlation and dNME-UC correlation were categorized as regions whose UC is independent of both NME and MML (Neither). The one-sided t-test was used to determine if there are more regions whose UC is predominantly correlated with NME change than regions whose UC is predominantly correlated with MML change. A one-sided t-test was chosen because visually there are more regions whose UC is predominantly correlated with NME than regions whose UC is predominantly correlated with MML. The same analysis using the other UC cutoffs, 0.025, 0.05, 0.15, 0.2 was done.

### Mouse Motif Analysis

736 human and mouse motifs from JASPAR were downloaded and the motifs were mapped to the mouse genome using CisGenome. Enrichment of each motif was calculated as the ratio between the odds of motif sites in the target regions (i.e. [number of target regions that contain motif sites]/[number of target regions that do not contain motif sites]) and the odds of motif sites in control regions (i.e., [number of control regions that contain motif sites]/[number of control regions that do not contain motif sites]). The control regions, which have a similar distribution of distance to TSS with the target regions, were obtained using CisGenome. One-sided Fisher's exact test was applied to test whether the motif is significantly enriched in the target regions. Multiple testing was adjusted by converting P-values to FDRs using Benjamini-Hochberg procedure. To compare the enrichment of each motif between regions where the methylation landscape change (i.e., UC) is predominantly correlated with NME with regions where UC is predominantly correlated with MML, a linear regression model was fitted to the normalized log odds ratio of motif enrichment from the two types of regions. The normalized log odds ratio was calculated using the log odds ratio divided by its standard error. TF motifs that were outside the 75% prediction interval were identified and were significantly enriched in either region type (FDR≤0.1). TF motifs that were more enriched in regions where UC is predominantly correlated with NME were marked with red color and defined as “entropy-associated motifs”, and TF motifs that are more enriched in regions where UC is predominantly correlated with MML were marked with blue color and defined as “mean-associated motifs”.

The entropy-associated TF motifs combined from all tissue types with TF motifs preferring high NME in the allele-specific analysis in human were also compared. By performing a one-sided Fisher's exact test, it was found that compared to mean-associated TF motifs, entropy-associated TF motifs in mouse have significantly higher overlap with the human high NME-allele preferring motifs. The one-sided Fisher's exact test was used because the assumption is the entropy-associated TF is more enriched in mouse than mean-associated TF. Similar to the analysis in humans, the relationship between NME and regulatory DNA in the mouse was studied. The NME between motif sites that are located in regulatory DNA as defined by a union set of mouse DNase I hypersensitive sites and those located in non-regulatory DNA (obtained similar to the human analysis) were compared in each mouse sample for 736 transcription factor binding motifs.

### Single-Cell RNA-Seq Data Processing

Human single-cell RNA-seq raw count data were downloaded from Human Cell Landscape (Microwell-seq platform). Cells with at least 500 expressed genes with non-zero read counts were retained. The read counts were normalized by library size and gene expression values were imputed using SAVER to address the high sparsity.

Mouse single-cell RNA-seq processed log 2-transformed FPKM data were downloaded from ENCODE (Fluidigm C1 SMART-seq platform). The log 2-transformed expression matrix was transformed back to the original scale and imputed using SAVER.

For both human and mouse data, SAVER imputed values were then log 2-transformed. Genes with non-zero expression in at least 10% of all cells were retained, and all ribosomal genes were removed. The processed data were used for the subsequent gene expression variability analysis.

### Calculation of Gene Expression Mean-Adjusted Variability (MAV)

Let yᵢⱼ be the library-size normalized, imputed, and log 2-transformed expression level for gene i (i=1, . . . , I) and cell j (j=1, . . . , J). Let mᵢ and sᵢ be the mean and standard deviation of the expression level for gene i across all cells respectively. A B-spline regression model was fitted across all genes where sᵢ is the response variable and mᵢ is the independent variable, and let gibe the fitted values of the standard deviations. The gene expression mean-adjusted variability (MAV) of gene i, hᵢ, is defined as the residual of the regression model, or equivalently the difference between observed and fitted standard deviation: hᵢ=sᵢ−ŝᵢ.

In addition, the BASiCS package that uses a Bayesian hierarchical model was ran to calculate residual overdispersion, which is mean-corrected gene expression variability on the same datasets. It was found that BASiCS and MAV provided similar results in both human (the Pearson correlation between NME and results from BASiCS near TSS is 0.21, the Pearson correlation between NME and MAV near TSS is 0.23,) and mouse (the Pearson correlation between NME and results from BASiCS near TSS is 0.11, the Pearson correlation between NME and MAV near TSS is 0.11). However, BASiCS requires spike-in or replicated samples. Most of our samples do not have spike-in or replicates. Thus, it was chosen to use the MAV calculation over BASiCS.

### Example 2

### Methylation Entropy can Depend on DNA Sequences

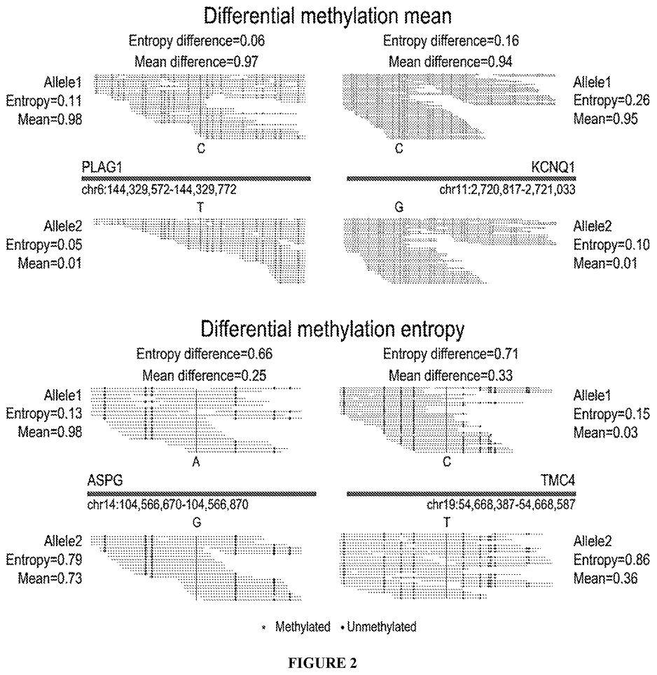

To identify DNA sequences specifically associated with methylation entropy, a recently developed information-theoretic method for allele-specific methylation analysis was applied to 49 human samples from the Roadmap Epigenomics Project. This approach allows to rigorously analyze genetic sequence-driven differences in methylation in the exact same cellular and tissue context. A methylation potential energy landscape model that considers all potential methylation states, cooperative interactions between adjacent sites, and adheres to the rigorous definition of Shannon entropy was used (see Example 1). In FIG. 2, four sets of sequencing reads are illustrated, where the two alleles are distinguished by a single nucleotide polymorphism (SNP), from which mean methylation level (MML) were calculated and methylation entropy (NME) normalized, i.e., normalized for the number of methylatable CpG sites. On the top are two genes, PLAG1 and KCNQ1 showing large mean differences, where one allele is much more methylated overall than the other. Explaining the large methylation difference between alleles, both PLAG1 and KCNQ1 are imprinted genes, i.e. with the parent of origin-specific expression, consistent with the known role of methylation in silencing imprinted loci on one allele. Indeed, regions with large mean allelic methylation difference were enriched in imprinted genes were found. Note that allelic differences of methylation in imprinted genes are not related to the underlying DNA sequence, the focus of the present study, because what is a maternal allele in one generation can be a paternal allele in the next. In contrast, at the bottom of FIG. 2 are examples of large sequence-driven differential methylation entropy between individual alleles at ASPG and TMC4, with much smaller differences in mean methylation levels than the examples on top. Note that previously sequence-independent entropy was studied in a way that does not separate two alleles, and such analysis identified imprinted genes as in our examples in FIG. 2 as if they had high methylation entropy even though they may not at allelic level. For example, for regions with the allelic difference in mean methylation like those in the top panel of FIG. 2, if one mixes the two alleles, then the entropy may appear to be higher in the allelic mixture, e.g., 0.28 for PLAG1, than in the individual alleles, but at the sequence level, this increase in entropy is actually due to parent of origin-specific imprinting. For each allele, the entropy is low. In examples such as the bottom of FIG. 2, the differences in entropy are specific to the DNA sequence (i.e., the entropy is high within an allele), which is the focus as the goal is to understand the underlying sequence drivers of entropy. Among the 3,332,744 regions containing heterozygous SNPs, 29,681 exhibited significant allele-specific differential NME were analyzed (FDR≤0.1), but only 6,807 regions exhibited significant differential MML (FDR≤0.1). Among those significant regions, 28,863 regions showed significant dNME without significant dMML and 5,989 regions showed significant dMML without significant dNME. Only 818 regions showed both significant dMML and dNME. This suggests that the methylation landscape change for a large proportion of regions can only be detected by dNME but not dMML. Indeed, it was found that dNME and dMML are not highly correlated, with a median R² of 0.07.

### Example 3

### Entropy-Associated DNA Sequences are Associated with Predicted Transcription Factor Binding Sites

The nucleotide sequence around each SNP was next scanned for transcription factor binding sites (TFBS) based on motifs from the JASPAR database and computed the binding probability in each allele. 129 motifs were identified with higher binding probability in the allele with a lower mean methylation level than the other allele, and only 7 motifs with higher binding probability in the allele with a higher mean methylation level than the other allele. Supporting the validity of the approach, CTCF was ranked 5th among the 129 motifs associated with low methylation levels, consistent with the known observation that the allele with higher CTCF binding probability shows lower methylation. Similarly, 89%-93% of motif sites of NFI family members, including NFIC, NFIB, NFIX, showed lower mean methylation level in the allele with higher binding probability than the other allele, consistent with the observation that the NFI family proteins are enriched at demethylated sites during neural development.

Using a similar approach, it was then asked what motifs are associated with allelic differences in methylation entropy. 135 motifs were found with higher binding probability in the allele with significantly higher methylation entropy than the other allele (binomial test, FDR≤0.1), compared to 14 motifs showing decreased binding probability in the allele with significantly higher methylation entropy at the same FDR level (binomial test, FDR≤0.1). Although there is no linear relationship between dMML and dNME, there is a slight negative correlation between dNME and dMML (median Pearson correlation=−0.18). Thus, the overlaps between high entropy-associated motifs and low MML-associated motifs were examined. Among these 135 higher entropy-associated motifs, 55 were not associated with low MML meaning they cannot be detected using MML (Table 1). Gene ontology (GO) enrichment analysis of the transcription factors associated with these 55 motifs shows enrichment for positive regulation of cell development (enrichment=5.43, P=4.72×10⁻⁵) and positive regulation of cell differentiation (enrichment=3.32, P=7.71×10⁻⁴). Furthermore, several of the transcription factors associated with these motifs are pioneer transcription factors, i.e., that open up condensed chromatin, including ASCL1, PBX1, MEIS1, ATF4, ESRRB, and KLF4. In contrast, low MML-associated motifs not associated with high NME showed no GO enrichment of the associated transcription factors.

Given that transcription factors generally bind to regulatory DNA to regulate their target genes, NME at regulatory DNA vs. non-regulatory DNA was then compared, defined using DNase I hypersensitive site sequencing data (see Methods and Materials). 583 out of 630 motifs whose motif sites in regulatory DNA showed significantly higher NME than those in non-regulatory DNA were identified (Wilcoxon's signed-rank test, FDR≤0.05). In contrast, the motifs sites in regulatory DNA showed lower MML than those in non-regulatory DNA for all motifs (Wilcoxon's signed-rank test, FDR≤0.05). These data also show that NME changes and MML changes between regulatory and non-regulatory regions do not always co-occur. GO analysis of transcription factors for motifs with higher NME in regulatory DNA was also highly enriched for developmental categories. Examples include the LHX5 motif, which regulates neuronal differentiation and dendritogenesis of Purkinje cells, and the SRF motif, which is required for vascular smooth muscle cell differentiation. Low MML was also associated with regulatory DNA, as expected (Wilcoxon's signed-rank test FDR≤0.05;), e.g. CTCF and NRF1

Table 1. The FDR is calculated from the P-values of the binomial test. The GO enrichment analysis for the ranked list of transcription factors whose motifs only have high NME is positive regulation of cell development (enrichment=5.43, P=4.72×10⁻⁵) and positive regulation of cell differentiation (enrichment=3.32, P=7.71×10⁻¹). Using the same GO enrichment analysis for the ranked list of transcription factors whose motifs only have low mml, there is no significantly enriched term using a P-value cutoff of 0.001.

TABLE 1
Transcription factors associated with high NME or low MML

| High NME-associated motifs not | | | | Low MML-associated motifs not | | | |
|---|---|---|---|---|---|---|---|
| associated with low MML | | | | associated with high NME | | | |
| TF | FDR | TF | FDR | TF | FDR | TF | FDR |
| MYOG | 4.14E−05 | TAL1::TCF3 | 4.75E−02 | LEF1 | 2.27E−05 | ELK1 | 1.65E−02 |
| SOX8 | 4.83E−05 | ATOH1(var. 2) | 4.92E−02 | ERF | 2.00E−04 | SOX9 | 1.65E−02 |
| ZNF148 | 2.33E−04 | SOX15 | 4.92E−02 | SP1 | 3.20E−04 | TCF21(var. 2) | 1.65E−02 |
| ASCL1 | 3.56E−04 | PBX2 | 4.92E−02 | JUN::JUNB | 4.06E−04 | ZBTB7A | 1.77E−02 |
| SOX10 | 5.73E−04 | PRDM4 | 4.92E−02 | JDP2 | 4.51E−04 | LHX9 | 2.11E−02 |
| MYF5 | 6.09E−04 | FOXK1 | 5.12E−02 | ELF5 | 8.87E−04 | MSGN1 | 2.19E−02 |
| FOXG1 | 6.52E−04 | TGIF2 | 5.25E−02 | ETV5 | 1.05E−03 | CEBPB | 3.05E−02 |
| CEBPA | 1.25E−03 | RARA::RXRA | 5.70E−02 | HNF4A(var. 2) | 1.11E−03 | CEBPE | 3.05E−02 |
| ZNF682 | 3.36E−03 | MEIS1(var. 2) | 5.96E−02 | SOX2 | 1.91E−03 | HMBOX1 | 3.73E−02 |
| MITF | 4.43E−03 | MSC | 6.96E−02 | FOXA2 | 2.37E−03 | RXRB | 4.22E−02 |
| BHLHA15(var. 2) | 4.82E−03 | ESRRB | 6.96E−02 | KLF11 | 3.40E−03 | TCF7L1 | 4.68E−02 |
| MAFF | 5.25E−03 | PKNOX1 | 6.96E−02 | KLF2 | 3.45E−03 | SP2 | 5.27E−02 |
| ZNF263 | 5.26E−03 | ZNF317 | 6.99E−02 | KLF3 | 3.45E−03 | TFAP4(var. 2) | 6.02E−02 |
| KLF4 | 5.46E−03 | JUND(var. 2) | 7.17E−02 | KLF15 | 4.89E−03 | RFX2 | 6.02E−02 |
| TGIF1 | 8.00E−03 | ATF2 | 7.20E−02 | TCF7 | 6.62E−03 | OTX2 | 7.38E−02 |
| RORA | 8.19E−03 | TEAD4 | 7.49E−02 | TCF7L2 | 7.94E−03 | GRHL2 | 7.38E−02 |
| PBX1 | 1.19E−02 | EWSR1-FLI1 | 8.33E−02 | SP9 | 8.41E−03 | FOXF2 | 8.40E−02 |
| FOXD2 | 1.23E−02 | JUN | 8.60E−02 | HNF4A | 1.00E−02 | ZNF410 | 8.67E−02 |
| KLF10 | 1.24E−02 | NHLH1 | 8.61E−02 | HNF4G | 1.00E−02 | TWIST1 | 8.99E−02 |
| RORB | 1.39E−02 | SOX14 | 8.61E−02 | IRF1 | 1.27E−02 | KLF14 | 9.73E−02 |
| FERD3L | 1.49E−02 | PKNOX2 | 8.61E−02 | GBX1 | 1.33E−02 | CEBPG | 9.76E−02 |
| RORC | 1.68E−02 | RARA::RXRG | 8.66E−02 | SP4 | 1.47E−02 | | |
| TBX4 | 1.70E−02 | POU2F1 | 8.66E−02 | ZBTB7B | 1.54E−02 | | |
| ZKSCAN5 | 2.70E−02 | TEAD2 | 9.04E−02 | FOXD1 | 1.54E−02 | | |
| FOXB1 | 3.29E−02 | POU2F2 | 9.23E−02 | FOXI1 | 1.54E−02 | | |
| POU3F4 | 3.81E−02 | PBX3 | 9.28E−02 | FOXO3 | 1.54E−02 | | |
| ATF4 | 4.18E−02 | PRRX2 | 9.42E−02 | FOXO6 | 1.54E−02 | | |
| POU1F1 | 4.57E−02 | | | NFATC4 | 1.65E−02 | | |

### Example 4

### Entropy is Inversely Related to CpG Density

it was then performed an allele-specific analysis to explore how normalized methylation entropy (NME) is related to DNA sequence other than transcription factor binding sites per se, by comparing allele-specific entropy to the trinucleotide DNA sequence contexts containing the given CpG dinucleotide (FIG. 3A, see Example 1). This analysis revealed several such contexts, but the strongest effects were seen where the SNP results in a lost CpG in one allele, and the allele with fewer CpG was 2.1-fold more likely to show higher NME than the alternate allele (FIG. 3A, Fisher's exact test, 95% CI [2.0-2.2], P<2.2×10⁻¹⁶) Comparing the number of CpG sites between the two alleles across all regions with significant allele-specific methylation entropy differences, the allele with more CpGs showed an average decrease in NME of 0.24 (FIG. 3B, one-sided paired t-test, P<2.2×10⁻¹⁶), supporting the observation that lower CpG number was associated with higher NME. NME across the genome within 20 kb of transcriptional start sites, regardless of allele, were further examined to include regions lacking SNPs. Here as well, there was an inverse correlation between NME and CpG density (FIG. 3C top panel, Pearson correlation test, correlation=−0.21, 95% CI=[−0.21, −0.21], P<2.2×10⁻¹⁶; see Example 1). A negative correlation was also observed between the mean methylation level and CpG density (FIG. 3C bottom panel, Pearson correlation test, correlation=−0.35, 95% CI=[−0.35,−0.35], P<2.2×10⁻¹⁶). MML showed a more abrupt decrease at high CpG density than did NME (FIG. 3C), consistent with the known compartmentalization of CpG islands.

### Example 5

### Information-Theory Based Analysis Reveals a Developmental Role of Entropy

To explore the developmental significance of methylation information content, mouse prenatal whole-genome bisulfite sequencing (WGBS) ENCODE3 data from 7 tissues at sequential embryonic developmental time points (E10.5 to E16.5) were analyzed, examining 46 samples with data for 1035 pairwise sample comparisons at each of 5,017,785 genomic regions. Postnatal time points were did not include since the environment of the animal is drastically different after birth, which could directly affect the epigenetic landscape, supported as well by the lack of a consistent developmental trajectory when a postnatal timepoint was included. For each pairwise comparison, the information-theoretic distance between two samples' methylation potential energy landscape, were calculated, quantified by the uncertainty coefficient (UC). UC measures the inherent information in a sample distinct from another sample, corrected for entropy (mathematically defined in Example 1).

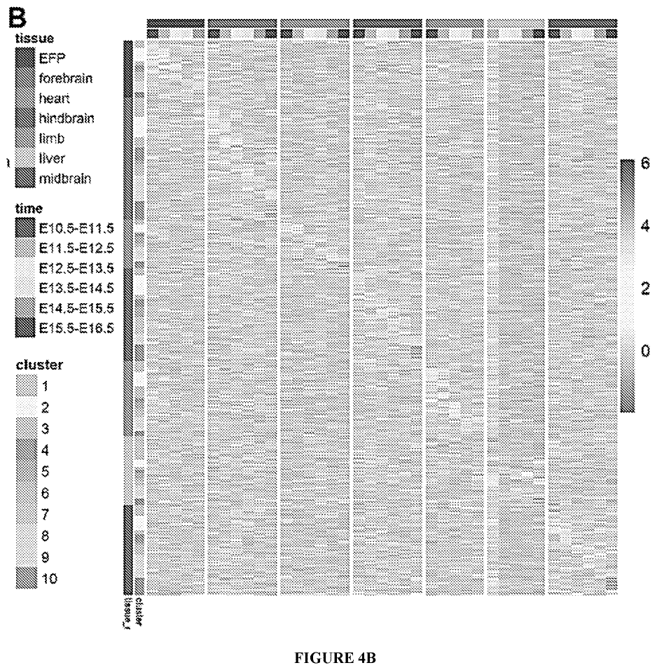

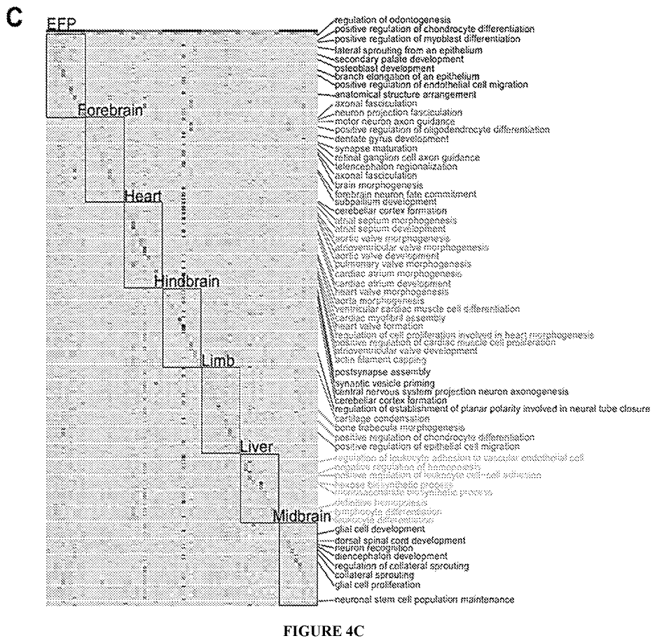

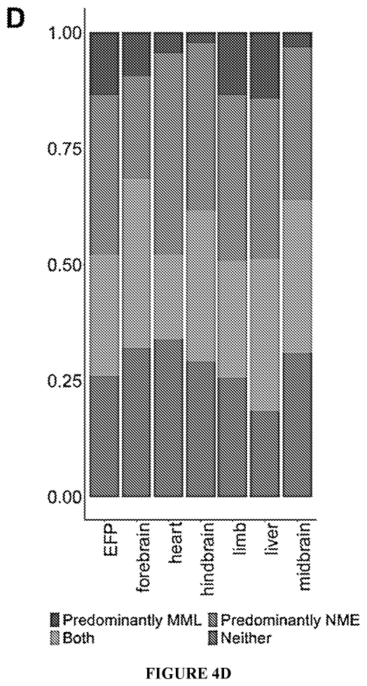

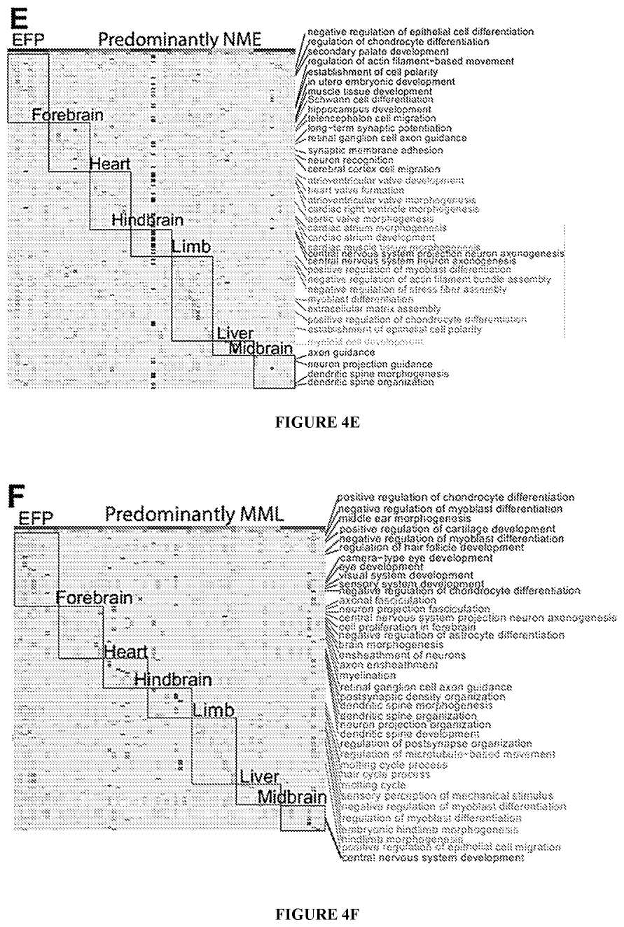

Multidimensional scaling (MDS) of all 1035 comparisons across the entire genome showed that samples from the same tissue were grouped together, while samples from different tissues were separated (FIG. 4A, top). Dissimilar tissue types, such as limb and liver, were separated from each other to a greater degree than similar tissue types, such as hindbrain, midbrain, and forebrain (FIG. 4A top). Furthermore, MDS showed that samples from the same tissue type were ordered based on developmental stage, with samples from earlier stages being closer to a common center across tissues, and samples from later stages being away from the center, progressing in different directions for different tissue types (FIG. 4A, bottom). Thus, the tissue-specific developmental trajectories can be derived from the information-theoretic content of DNA methylation.

Clustering analysis was then performed within each tissue in order to determine whether there was a specific set of genomic regions undergoing methylation landscape changes at each developmental stage, and if so, whether those time-specific clusters were also specific to the given tissue in which they were observed. To do this, for each tissue, clustering analysis was performed on all pairs of adjacent time points from earliest to latest, analyzing 2,085,884 regions showing large methylation landscape change (defined by UC>0.1 in at least one tissue between any two time points, see Example 1) for each comparison. The ordering of the regions in the clustering heatmap recaptured the developmental process, confirming that regions with high UC capture developmental change. The temporal patterns were tissue-specific, as clustering patterns observed in one tissue were not observed in other tissues. These tissue-specific regions with high UC also captured time-dependent developmental changes (FIG. 4B). Furthermore, these developmental clusters in a given tissue were almost entirely specific to that tissue (FIG. 4B). UC>0.1 corresponded to the top 5% of all UC values in the entire dataset. Using other UC cutoffs from 0.025 to 0.2, the tissue-specific clustering patterns were still observed.

Next, it was asked whether the genes linked to these developmental- and tissue-specific regions are functionally related to those tissues by GO analysis. The regions were mapped to regulatory elements of specific genes, including promoters (within 2 kb of the transcriptional start site), and enhancers as described. Enhancers showed substantially larger changes in both NME and MML than promoters, so enhancer regions were initially focused on (FIG. 4C). The GO terms identified in this manner were strikingly tissue-specific as well as specific to each temporal cluster, and highly related to the development function of each tissue as well. For example, GO terms related to embryonic facial prominence (EFP), such as positive regulation of chondrocyte differentiation, showed large and significant enrichment only in EFP and not in other tissues; and GO terms related to liver, such as monosaccharide biosynthetic process, showed large and significant enrichment only in liver and not in other tissues (FIG. 4C). Moreover, in some tissues, the temporal order of GO terms also reflected the temporal developmental program (forebrain and heart, see FIG. 4C). Using the same method with other UC cutoffs from 0.025 to 0.2, significant tissue- and time-specific GO enrichment were still found.

Although the focus was on enhancer regions, as explained above, the genes whose promoters showed significant methylation landscape change were also analyzed. It was found that those genes were enriched in fewer tissue-specific terms than genes with methylation landscape changes in enhancer regions. This was consistent with the finding that enhancers showed larger NME and MML changes than promoters, confirming that epigenetic landscape changes at enhancers were more strongly related to tissue-specific development.

### Example 6

### Uc Shows Comparable or Stronger Correlation with Entropy Compared to Mean Methylation

UC measures methylation landscape changes between samples and captures both mean methylation changes and methylation entropy changes. To determine the relative contribution of mean methylation and methylation entropy change to the developmental landscape distance as measured by UC as described above, the correlation between MML change and NME change to UC was next calculated, classifying methylation changes at each region as predominantly correlated with MML, NME, both, or neither (see Example 1). In the regions with UC>0.1 between developmental timepoints, UC was predominantly correlated with MML change in 2%-14%, depending on the tissue, with NME change in 22-43%, with both MML and NME change in 18-36% and with neither in 19%-34% regions (FIG. 4D). These data suggested that in all seven tissues, the contribution of NME change to UC is at least comparable to, if not larger than, the contribution of MML change to UC. Even using other UC cutoffs from 0.025 to 0.2, similar trend in development was still observed.

To understand the biological function of those regions with different categories as shown in FIG. 4D, a GO annotation analysis was performed on the genes with enhancers in the four UC-correlation categories described above. Predominantly NME-correlated regions have a slightly larger number of enriched GO terms for tissue-specific functions compared to predominantly MML-correlated regions (FIGS. 4E and 4F). Given that these two sets of regions are non-overlapping, it shows that NME and MML each contains unique information about tissue-specific functions not captured by the other. There was also substantial enrichment for tissue-specific functions in UC regions correlated with both mean and entropy change, and UC also captured some information independent of both mean and entropy change. Regions were also annotated based on whether they are linked to tissue-specific genes, and it was found that the predominantly NME-correlated regions and the predominantly MML-correlated regions covered similar numbers of tissue-specific genes. These results demonstrated that methylation entropy contains at least as much unique information about the developmental epigenetic landscape as does mean methylation. Thus, analyzing only mean methylation prevents one from identifying a substantial fraction of DNA methylation landscape changes characterized by the information-theoretic measure UC.

### Example 7

### Developmental Entropy and Mean are Associated with Different Transcription Factor Binding Sites

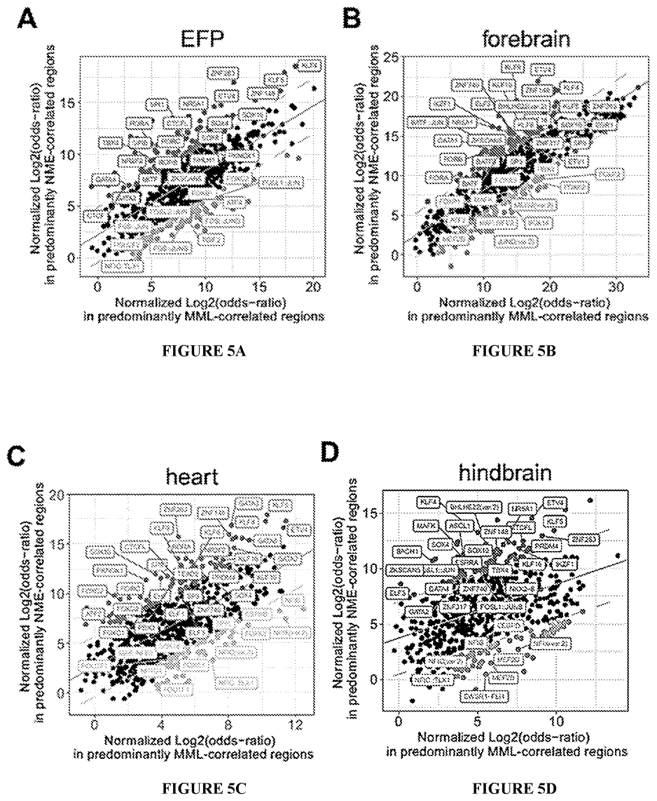

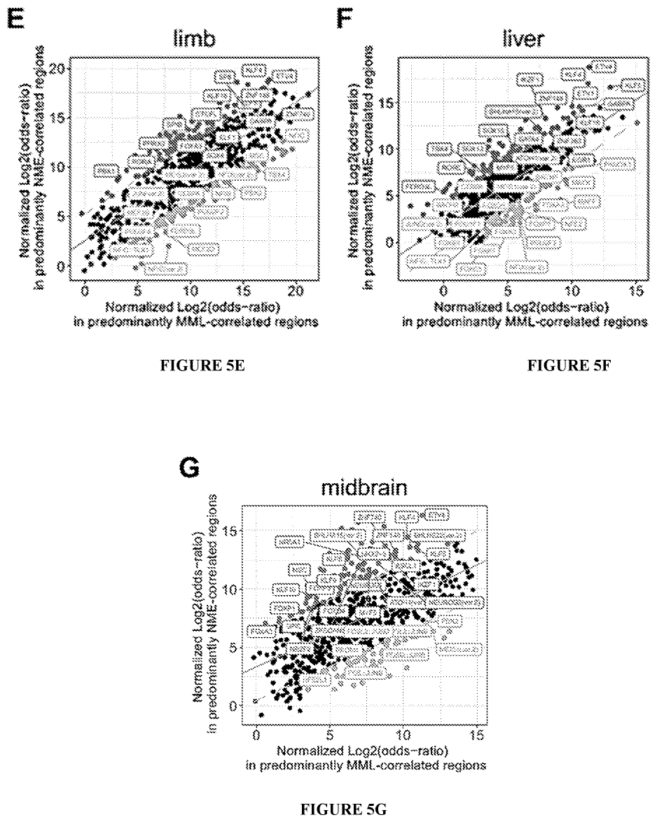

Given the results above showing that mean and entropy were associated with different transcription factor motifs in human, and also associated with different genomic regions based on embryonic development, it was asked whether different transcription factor motifs are associated with mean- or entropy-related developmental methylation changes in the mouse. Regions where UC was predominantly correlated with NME change, were compared to regions where UC was predominantly correlated with MML change (FIG. 5, see Example 1). Different sets of motifs were found to be associated with entropy versus mean. In total, 344 entropy-associated motifs and 324 mean-associated motifs were identified. FIG. 5 shows the enrichment of each motif in the regions where UC was predominantly correlated with NME change versus the regions where UC was predominantly correlated with MML change in all 7 tissues analyzed. For example, KLF family motifs such as KLF4 and KLF5 are among the entropy-associated motifs which appear in multiple tissue types such as heart, forebrain, limb, EFP, midbrain, hindbrain, and liver (FIGS. 5A-5G). KLF4 is known to be a key transcription factor in embryonic development and is also one of the four transcription factors to generate iPSC. Among mean-associated motifs, NF-I family motifs such as NFIC, NFIX, and NFIB were identified in multiple tissues such as heart, limb, EFP, hindbrain, and liver (FIGS. 5A, 5C, 5D, 5E, and 5F. Mutations in Nfi family members are associated with brain development defects. Using entropy-associated motifs, enhancers with entropy changes were annotated as well as their target genes (Table 2), and similarly for enhancers with mean methylation changes. For instance, Celsr2, which has been shown to regulate forebrain wiring, showed a large entropy change between E12.5 and E13.5 at its enhancer, which is also the KLF4 transcription factor binding site. Similarly, Gata5 plays a critical role in heart development and Gata5 knockout mice develop bicuspid aortic valve. Gata5 showed a large entropy change between E11.5 and E12.5 at its enhancer, which is also the GLI3 binding site. These data suggest that entropy-associated transcription factor binding may play an important role in modulating enhancer function in development.

Table 2. Genes whose enhancers contain entropy-associated motifs, are listed in the “Entropy-associated TF motif” column. At the enhancer of those genes, the correlations between UC and NME difference (dNME) are much greater than the correlation between UC and MML difference (dMML) and the dNME is much larger than dMML. Those genes are known to be functional in the corresponding tissue from the literature listed in the PMID column. This suggests that those entropy-associated motifs are at the enhancers of functional genes.

TABLE 2
Genes with enhancers contain entropy-associated transcription factor motifs

| | Predicted | | | | dNME- | | dMML- | | |
|---|---|---|---|---|---|---|---|---|---|
| Tissue | Motif | Cluster | Gene | dNME | UC cor | dMML | UC cor | Stage | PMID |
| EFP | NR5A1; | 4 | Edn1 | 0.53 | 0.98 | 0.05 | −0.26 | E12.5- | 24268655; |
| | NR2F2 | | | | | | | E13.5 | 25772936 |
| | NR2F2 | 10 | Hat1 | 0.50 | 0.79 | 0.0 | −0.80 | E14.5- | 23754951 |
| | | | | | | | | E15.5 | |
| | ZNF263; | 2 | Trps1 | 0.45 | 0.97 | 0.22 | 0.74 | E11.5- | 22581230 |
| | CTCFL; | | | | | | | E12.5 | |
| | GATA2 | | | | | | | | |
| | NR5A1; | 2 | Tgfbr2 | 0.43 | 0.95 | 0.01 | −0.60 | E11.5- | 15731757 |
| | ETV4; | | | | | | | E12.5 | |
| | NR2F2 | | | | | | | | |
| | GATA2 | 1 | Alx4 | 0.35 | 0.77 | 0.01 | 0.26 | E13.5- | 11137991 |
| | | | | | | | | E14.5 | |
| Forebrain | KLF5 | 5 | Prdm16 | 0.29 | 0.66 | 0.01 | −0.21 | E15.5- | 28698301 |
| | | | | | | | | E16.5 | |
| | IKZF1; | 8 | Otx2 | 0.41 | 0.95 | 0.04 | −0.47 | E14.5- | 1353865; |
| | ELF3; | | | | | | | E15.5 | 14625556 |
| | ETV1 | | | | | | | | |
| | KLF4; | 10 | Celsr2 | 0.37 | 0.81 | 0.06 | −0.06 | E15.5- | 25002511 |
| | KLF5 | | | | | | | E16.5 | |
| | IKZF1; | 10 | Nfia | 0.43 | 0.86 | 0.13 | 0.17 | E15.5- | 12514217 |
| | ETV1; | | | | | | | E16.5 | |
| | SOX10; | | | | | | | | |
| | NR5A1 | | | | | | | | |
| | ZNF740; | 2 | Nfib | 0.40 | 0.97 | 0.01 | −0.02 | E11.5- | 27965439 |
| | KLF4; | | | | | | | E12.5 | |
| | KLF5; | | | | | | | | |
| | KLF6 | | | | | | | | |
| limb | PBX1 | 9 | Kat6b | 0.60 | 0.99 | 0.08 | −0.06 | E13.5- | 22265014; |
| | | | | | | | | E14.5 | 22077973 |
| | SPIB; | 2 | Ror2 | 0.25 | 0.76 | 0.07 | −0.17 | E11.5- | 20660756; |
| | ETV4 | | | | | | | E12.5 | 21316585; |
| | | | | | | | | | 10700182 |
| | ZNF740 | 10 | Ski | 0.49 | 0.97 | 0.15 | 0.66 | E14.5- | 12435627 |
| | | | | | | | | E15.5 | |
| | ZNF740; | 2 | Irx3 | 0.49 | 0.79 | 0.05 | −0.39 | E10.5- | 24726282 |
| | SPIB | | | | | | | E11.5 | |
| | ETV4 | 2 | Wnt5a | 0.45 | 0.97 | 0.04 | −0.13 | E10.5- | 21316585 |
| | | | | | | | | E11.5 | |
| heart | KLF5; | 8 | Gata4 | 0.51 | 0.80 | 0.15 | 0.56 | E14.5- | 27984724; |
| | KLF4; | | | | | | | E15.5 | 22522929; |
| | ETV4; | | | | | | | | 26875865; |
| | KLF9; SP8; | | | | | | | | 31080136 |
| | ZNF148; | | | | | | | | |
| | PRDM4; | 10 | Ppif | 0.44 | 0.96 | 0.02 | −0.32 | E15.5- | 15800627; |
| | SOX4 | | | | | | | E16.5 | 20890047 |
| | PBX3; | 4 | Ror1 | 0.46 | 0.87 | 0.15 | 0.62 | E11.5- | 30342492; |
| | PKNOX1; | | | | | | | E12.5 | 11713269 |
| | SOX10 | | | | | | | | |
| | KLF6; | 8 | Ndrg2 | 0.62 | 1.00 | 0.20 | 0.75 | E14.5- | 22523601; |
| | TEAD2; | | | | | | | E15.5 | 16520977 |
| | RORC | | | | | | | | |
| | ETV4; | 8 | Stat6 | 0.59 | 0.99 | 0.07 | −0.15 | E13.5- | 33192584; |
| | ATF2 | | | | | | | E14.5 | 25722436 |

### Example 8

### Entropy-Associated Sequence and its Association with Regulatory DNA are Conserved Between Human and Mouse

Next, it was asked whether the entropy-associated sequence and chromatin features are conserved between human and mouse. The percentage of overlap between human motifs associated with high NME and mouse entropy-associated motifs was significantly higher than the percentage of overlap between the human motifs associated with high NME and the mouse MML-related motifs (Fisher's exact test, odds ratio=1.43, P=0.04). The mouse entropy-associated motifs that overlap with the human motifs preferring high NME are highlighted in blue boxes in FIG. 5. Those entropy-associated motifs can play a critical role in the corresponding tissue, such as PBX3, whose entropy-associated binding motifs are conserved in human and mouse, and are associated with heart development but not forebrain and limb, is necessary for normal heart development, PKNOX1, which is associated with congenital heart defects and FOXC1, which is required for cardiovascular development. In the forebrain, SOX10, SP8, and RORA were found (FIG. 5B), which regulate myelination-related genes, promote olfactory bulb interneurons during the development, and regulate genes associated with autism spectrum disorder. In limb, SOX4 was found, which is related to massive cartilage fusion, ETV4, associated with sonic hedgehog pathways in limb outgrowth, and PBX1, required for chondrocyte proliferation.

Similar conservation was found between mouse and human for the relationship of entropy to CpG density and regulatory DNA. For CpG density, similar to human (FIG. 3C), a strong correlation was observed between local CpG density and NME in mouse samples (Pearson correlation test, correlation=−0.21, 95% CI=[−0.21,−0.21], P<2.2×10⁻¹⁶). To explore the relationship between NME and transcription factor motif binding sites at regulatory DNA in mouse, the NME between motif sites that are in regulatory DNA as defined by a union set of mouse DNase I hypersensitive sites and those located in non-regulatory DNA in each mouse sample for 736 TF motifs wree compared. Similar to human motif analysis, it was found that most of the motifs in mouse have significantly higher NME in regulatory DNA than non-regulatory DNA (661 motifs with FDR≤0.05). Whether those motifs with high NME in the regulatory region are conserved between humans and mice was further examined. In the tissues that exist in both human and mouse, it was found that 79% (583 motifs) of the motifs show higher NME in regulatory regions than non-regulatory regions in both human and mouse. In the tissues that only exist in one species, 81.5% (600 motifs) of the motifs show higher NME in regulatory regions than in non-regulatory regions were observed. This suggests that the observation of high NME in regulatory regions is largely tissue-independent.

Together, these results showed that the relationships between NME and transcription factor binding, CpG density, and regulatory DNA were highly conserved between mouse and human, thus supporting their functional significance.

### Example 9

### Methylation Entropy is Associated with Gene Expression Variability in Both Human and Mouse

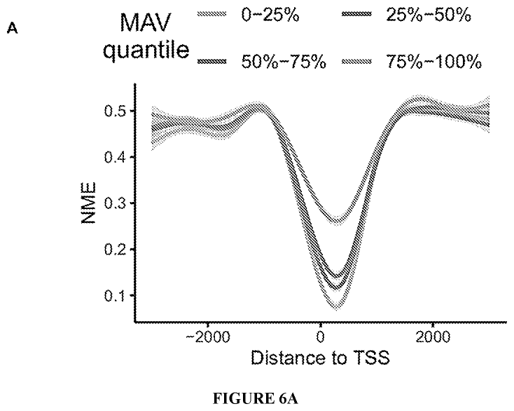

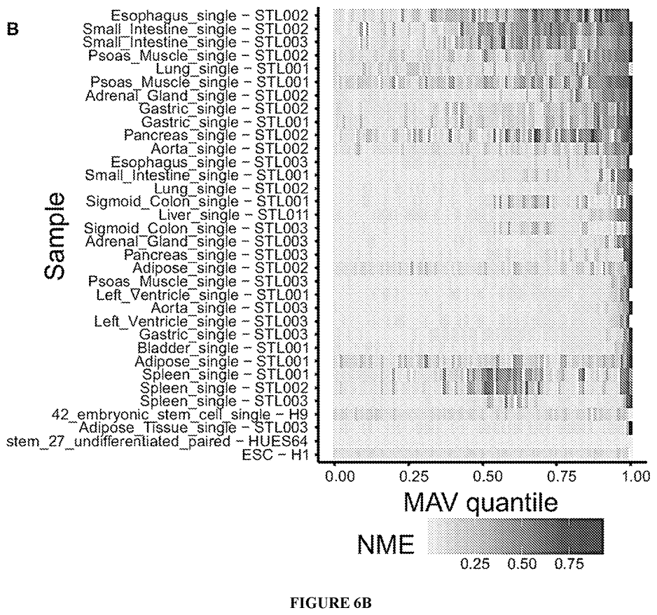

Because NME is associated with expression variability in human cancer cells, it was asked whether methylation entropy is related to gene expression variability in normal human somatic tissues and mouse embryonic development. First, single-cell RNA-seq data from 14 different human somatic tissues and human ESC that were already done methylation analysis from WGBS data for the corresponding tissues were re-analyzed (see Example 1 for details). A correlation between NME near the transcription start site (TSS) of the genes and mean-adjusted variability (MAV) of gene expression was found (see Example 1). Genes with higher MAV tended to have larger NME near TSS while genes with lower MAV tended to have smaller NME near TSS (FIG. 6A). The correlation between the NME close to the TSS and the gene expression variability was highly significant (Pearson correlation test, average correlation=0.23, average 95% CI=[0.21, 0.25], average P=7.6×10⁻⁵). A similar pattern was observed in other human samples except for undifferentiated stem cell lines (FIG. 6B). Thus, NME was generally positively correlated with gene expression variability but not mean expression.

Given the enrichment of regions with large MML difference in imprinted genes, the relationship between MML and monoallelic gene expression (MAE) was explored. A significant enrichment of the regions with large allelic MML difference in the promoters of MAE genes was observed (Fisher's exact test, odds ratio=1.49, 95% CI=[1.04-2.15], P=0.03). In contrast, as expected, no enrichment of the regions with large allelic NME difference were observed (Fisher's exact test, odds ratio=1.03, 95% CI=[0.79-1.35], P=0.84) suggesting no association between methylation entropy and mean gene expression. Thus, while mean methylation is associated with mean expression, entropic methylation is associated with expression variability (MAV).

The relationship between NME and MAV in mouse was next analyzed, analyzing mouse fetal limb tissue scRNA-seq data from the ENCODE3 project and calculating MAV in the same way as in human. Consistent with the human analysis, genes with higher MAV showed larger NME near TSS while genes with lower MAV showed smaller NME near TSS, and there was a smaller but still statistically significant correlation between NME and MAV (Pearson correlation test, average correlation=0.11, average 95% CI=[0.10, 0.11], average P<2.2×10⁻¹⁶. Those observations showed that the association of NME and gene expression variability in normal tissue is also conserved between human and mouse.

### Example 10

### Discussion

In summary, a systematic characterization of DNA methylation entropy has been performed, relating it to DNA sequence, predicted transcription factor binding sites, regulatory DNA, its relative contribution to epigenetic landscape changes during mouse development, and its relationship to variable gene expression. Applying an information-theoretic approach to mouse embryo DNA methylation data, tissue- and time-specific transitions that are much more discriminative than conventional mean-based DNA methylation analyses were identified, identifying temporal transitions remarkably well and essentially uniquely for each tissue type. By using the information metric of UC, the relative contribution of differences in entropy versus mean methylation to these informational changes in the epigenetic landscape of mouse development could also be directly compared. In all tissues, it was observed more regions where UC is predominantly correlated with NME change (FIG. 4D, 22%-43%) than the regions where UC is predominantly correlated with MML change (FIG. 4D, 2%-14%). This, along with the gene ontology enrichment analysis (FIGS. 4C, 4E, and 4F) and additional analysis of tissue-specific genes suggests that the role of methylation entropy in shaping the dynamic changes of methylation landscape is comparable to, if not larger than, mean methylation. Gene ontology enrichment analysis supported a functional role of these entropy changes, particularly at enhancers, identifying many known critical genes for development in those tissues. These specific functional categories were often congruent to the functional development of those tissues, as well, as evidenced particularly in the heart.

Certain DNA sequence relationships with methylation entropy were also identified, using allele-specific analysis comparing directly the entropy or mean of sequences differing by a single nucleotide in the same cells. There were approximately 3-fold more entropy- than mean-distinguishing SNPs. Moreover, these sequence differences were directly related to changes in many transcription factor binding sites. Note that imprinted genes were associated with differential mean methylation between alleles, although if the alleles are pooled they will appear entropic. There was also a strong inverse relationship of entropy with small differences in CpG density, in contrast to the comparatively weak inverse association of CpG density with mean methylation except at very high-density islands.

Moreover, entropic methylation was strongly associated with gene expression variability in human normal somatic cells and normal mouse development, in contrast to the monoallelic expression which was associated with differential mean methylation as seen here and elsewhere. The fact that so many entropy-associated features were found to be conserved between human and mouse suggests that they serve an important functional role. This relationship supports a potential role of epigenetic entropy in mediating either buffering or phenotypic divergence, two sides of the same developmental coin. The link between epigenetic entropy and the sequence of specific transcription factor binding sites suggests that binding of some transcription factors may regulate a variable response to environmental signaling, since many of these transcription factors are signaling-dependent, as classified by Brivanlou and Darnell. These include nuclear receptor family members such as NR5A1, RORA, RORB, and accessory factors like KLF's, and receptor-ligand targets including ETS-family members (ETV's, ELF's), receptor tyrosine kinases like EGFRs, and HIPPO-signaling targets like TEADs, all of which were conserved entropy-associated binding regions between human and mouse.

A limitation of the mouse data analysis is inherent in the datasets from which they were derived (and as those authors also acknowledged as a limitation), namely that a given tissue sample would include multiple cell types which could affect inferences about developmental changes. This admixture would likely affect conclusions regarding entropy changes overtime in development. However, entropy itself likely does contribute meaningfully to developmental transitions based on the current analysis for two reasons. First, the regions associated with high entropy change are located at the enhancers of the genes that are specific to the development of corresponding tissue as shown in the gene ontology (GO) analysis. Second, this limitation of admixture does not apply to the human data sets since in that case entropy differences were at the level of individual alleles. Moreover, the entropy-associated transcription factor motifs were conserved between the human data set and the mouse embryonic data set.

It will be interesting to explore the relationship between polymorphisms and epigenetic entropy in large cohorts, which could not be done on the relatively small number of human samples that were analyzed here, since rare variants in transcription factor binding sites have been associated with altered methylation at specific CpG sites in human lymphocytes. Previously, Mendelian transmission of 383 regions with variable mean methylation in the human genome were identified, and two recent studies using GTEx data show SNPs associated with variation in mean methylation in a tissue-specific manner. An alternative approach would be to analyze genetically complex mouse strains like the collaborative cross, which was not possible in the inbred mouse strain studied here; and this mouse approach would allow for hypothesis testing of specific transcription factor engineered mutations in the development.

Finally, the results shown here emphasize the importance of including entropy analysis in genome-wide studies of DNA methylation. While this has been widely adopted in some human diseases such as cancer, the role of methylation entropy in embryonic development may be under-appreciated. A recent study supports that idea, showing that knockout of DNA methyltransferases increases methylation entropy and gene expression variability in cultured embryonic stem cells. The results presented here identify genetic sequences and their associated motifs underlying epigenetic entropy and their critical role in development.

### REFERENCE

1. Hannon, E., Gorrie-Stone, T. J., Smart, M. C., Burrage, J., Hughes, A., Bao, Y., Kumari, M., Schalkwyk, L. C. and Mill, J. (2018) Leveraging DNA-Methylation Quantitative-Trait Loci to Characterize the Relationship between Methylomic Variation, Gene Expression, and Complex Traits. Am J Hum Genet, 103, 654-665.
2. Anastasiadi, D., Esteve-Codina, A. and Piferrer, F. (2018) Consistent inverse correlation between DNA methylation of the first intron and gene expression across tissues and species. Epigenetics Chromatin, 11, 37.
3. Feinberg, A. P., Koldobskiy, M. A. and Gondor, A. (2016) Epigenetic modulators, modifiers and mediators in cancer aetiology and progression. Nat Rev Genet, 17, 284-299.
4. Pujadas, E. and Feinberg, A. P. (2012) Regulated noise in the epigenetic landscape of development and disease. Cell, 148, 1123-1131.
5. Landan, G., Cohen, N. M., Mukamel, Z., Bar, A., Molchadsky, A., Brosh, R., Horn-Saban, S., Zalcenstein, D. A., Goldfinger, N., Zundelevich, A. et al. (2012) Epigenetic polymorphism and the stochastic formation of differentially methylated regions in normal and cancerous tissues. Nat Genet, 44, 1207-1214.
6. Jenkinson, G., Pujadas, E., Goutsias, J. and Feinberg, A. P. (2017) Potential energy landscapes identify the information-theoretic nature of the epigenome. Nat Genet, 49, 719-729.
7. Tsankov, A. M., Wadsworth, M. H., 2nd, Akopian, V., Charlton, J., Allon, S. J., Arczewska, A., Mead, B. E., Drake, R. S., Smith, Z. D., Mikkelsen, T. S. et al. (2019) Loss of DNA methyltransferase activity in primed human ES cells triggers increased cell-cell variability and transcriptional repression. Development, 146.
8. Abante, J., Fang, Y., Feinberg, A. P. and Goutsias, J. (2020) Detection of haplotype-dependent allele-specific DNA methylation in WGBS data. Nat Commun, 11, 5238.
9. Kundaje, A., Meuleman, W., Ernst, J., Bilenky, M., Yen, A., Heravi-Moussavi, A., Kheradpour, P., Zhang, Z., Wang, J., Ziller, M. J. et al. (2015) Integrative analysis of 111 reference human epigenomes. Nature, 518, 317-330.
10. He, Y., Hariharan, M., Gorkin, D. U., Dickel, D. E., Luo, C., Castanon, R. G., Nery, J. R., Lee, A. Y., Zhao, Y., Huang, H. et al. (2020) Spatiotemporal DNA methylome dynamics of the developing mouse fetus. Nature, 583, 752-759.
11. Onuchic, V., Lurie, E., Carrero, I., Pawliczek, P., Patel, R. Y., Rozowsky, J., Galeev, T., Huang, Z., Altshuler, R. C., Zhang, Z. et al. (2018) Allele-specific epigenome maps reveal sequence-dependent stochastic switching at regulatory loci. Science, 361.
12. Martin, M. (2011) Cutadapt removes adapter sequences from high-throughput sequencing reads. EMBnet. journal, 17, 10-12.
13. Wilton, R., Li, X., Feinberg, A. P. and Szalay, A. S. (2018) Arioc: GPU-accelerated alignment of short bisulfite-treated reads. Bioinformatics, 34, 2673-2675.
14. Brouard, J. S., Schenkel, F., Marete, A. and Bissonnette, N. (2019) The GATK joint genotyping workflow is appropriate for calling variants in RNA-seq experiments. J Anim Sci Biotechnol, 10, 44.
15. Sherry, S. T., Ward, M. H., Kholodov, M., Baker, J., Phan, L., Smigielski, E. M. and Sirotkin, K. (2001) dbSNP: the NCBI database of genetic variation. Nucleic Acids Res, 29, 308-311.
16. Benjamini, Y. and Hochberg, Y. (1995) Controlling the false discovery rate: a practical and powerful approach to multiple testing. Journal of the Royal statistical society: series B (Methodological), 57, 289-300.
17. Jirtle, R. (2012) Imprinted Genes: by Species, Geneimprint
18. Savova, V., Chun, S., Sohail, M., McCole, R. B., Witwicki, R., Gai, L., Lenz, T. L., Wu, C. T., Sunyaev, S. R. and Gimelbrant, A. A. (2016) Genes with monoallelic expression contribute disproportionately to genetic diversity in humans. Nat Genet, 48, 231-237.
19. Fornes, O., Castro-Mondragon, JA., Khan, A., van der Lee, R., Zhang, X., Richmond, P.A., Modi, B. P., Correard, S., Gheorghe, M., Baranasid, D. et al. (2020) JASPAR 2020: update of the open-access database of transcription factor binding profiles. Nucleic Acids Res, 48, D87-d92.
20. Saxonov, S., Berg, P. and Brutlag, D. L. (2006) A genome-wide analysis of CpG dinucleotides in the human genome distinguishes two distinct classes of promoters. Proc Natl Acad Sci USA, 103, 1412-1417.
21. Ji, H., Jiang, H., Ma, W., Johnson, D. S., Myers, R. M. and Wong, W. H. (2008) An integrated software system for analyzing ChIP-chip and ChIP-seq data. Nat Biotechnol, 26, 1293-1300.
22. Consortium, E. P. (2012) An integrated encyclopedia of DNA elements in the human genome. Nature, 489, 57.
23. Zhou, W., Ji, Z., Fang, W. and Ji, H. (2019) Global prediction of chromatin accessibility using small-cell-number and single-cell RNA-seq. Nucleic Acids Res, 47, e121.
24. Gorkin, D.U., Barozzi, I., Zhao, Y., Zhang, Y., Huang, H., Lee, A. Y., Li, B., Chiou, J., Wildberg, A., Ding, B. et al. (2020) An atlas of dynamic chromatin landscapes in mouse fetal development. Nature, 583, 744-751.
25. Alexa, A. and Rahnenführer, J. (2009) Gene set enrichment analysis with topGO. Bioconductor Improv, 27, 1-26.
26. Han, X., Zhou, Z., Fei, L., Sun, H., Wang, R., Chen, Y., Chen, H., Wang, J., Tang, H., Ge, W. et al. (2020) Construction of a human cell landscape at single-cell level. Nature, 581, 303-309.
27. Huang, M., Wang, J., Torre, E., Dueck, H., Shaffer, S., Bonasio, R., Murray, J. I., Raj, A., Li, M. and Zhang, N. R. (2018) SAVER: gene expression recovery for single-cell RNA sequencing. Nat Methods, 15, 539-542.
28. He, P., Williams, B. A., Trout, D., Marinov, G. K., Amrhein, H., Berghella, L., Goh, S. T., Plajzer-Frick, I., Afzal, V., Pennacchio, L. A. et al. (2020) The changing mouse embryo transcriptome at whole tissue and single-cell resolution. Nature, 583, 760-767.
29. Eling, N., Richard, A. C., Richardson, S., Marioni, J. C. and Vallejos, C. A. (2018) Correcting the Mean-Variance Dependency for Differential Variability Testing Using Single-Cell RNA Sequencing Data. Cell Syst, 7, 284-294 e212.
30. Iglesias-Platas, I., Court, F., Camprubi, C., Sparago, A., Guillaumet-Adkins, A., Martin-Trujillo, A., Riccio, A., Moore, G. E. and Monk, D. (2013) Imprinting at the PLAGLI domain is contained within a 70-kb CTCF/cohesin-mediated non-allelic chromatin loop. Nucleic Acids Res, 41, 2171-2179.
31. Umlauf, D., Goto, Y., Cao, R., Cerqueira, F., Wagschal, A., Zhang, Y. and Feil, R. (2004) Imprinting along the Kcnql domain on mouse chromosome 7 involves repressive histone methylation and recruitment of Polycomb group complexes. Nat Genet, 36, 1296-1300.
32. Li, E., Beard, C. and Jaenisch, R. (1993) Role for DNA methylation in genomic imprinting. Nature, 366, 362-365.
33. Monk, D., Mackay, D. J. G., Eggermann, T., Maher, E. R. and Riccio, A. (2019) Genomic imprinting disorders: lessons on how genome, epigenome and environment interact. Nat Rev Genet, 20, 235-248.
34. Coetzee, S. G., Coetzee, G. A. and Hazelett, D. J. (2015) motifbreakR: an R/Bioconductor package for predicting variant effects at transcription factor binding sites. Bioinformatics, 31, 3847-3849.
35. Bell, A. C. and Felsenfeld, G. (2000) Methylation of a CTCF-dependent boundary controls imprinted expression of the Igf2 gene. Nature, 405, 482-485.
36. Sanosaka, T., Imamura, T., Hamazaki, N., Chai, M., Igarashi, K., Ideta-Otsuka, M., Miura, F., Ito, T., Fujii, N., Ikeo, K. et al. (2017) DNA Methylome Analysis Identifies Transcription Factor-Based Epigenomic Signatures of Multilineage Competence in Neural Stem/Progenitor Cells. Cell Rep, 20, 2992-3003.
37. Eden, E., Navon, R., Steinfeld, I., Lipson, D. and Yakhini, Z. (2009) GOrilla: a tool for discovery and visualization of enriched GO terms in ranked gene lists. BMC Bioinformatics, 10, 48.
38. Park, N. I., Guilhamon, P., Desai, K., McAdam, R. F., Langille, E., O'Connor, M., Lan, X., Whetstone, H., Coutinho, F. J., Vanner, R. J. et al. (2017) ASCL1 Reorganizes Chromatin to Direct Neuronal Fate and Suppress Tumorigenicity of Glioblastoma Stem Cells. Cell Stem Cell, 21, 209-224.e207.
39. Sun, Y., Zhou, B., Mao, F., Xu, J., Miao, H., Zou, Z., Phuc Khoa, L. T., Jang, Y., Cai, S., Witkin, M. et al. (2018) HOXA9 Reprograms the Enhancer Landscape to Promote Leukemogenesis. Cancer Cell, 34, 643-658.e645.
40. Adachi, K., Kopp, W., Wu, G., Heising, S., Greber, B., Stehling, M., Aranzo-Bravo, M. J., Boerno, S. T., Timmermann, B., Vingron, M. et al. (2018) Esrrb Unlocks Silenced Enhancers for Reprogramming to Naive Pluripotency. Cell Stem Cell, 23, 266-275.e266.
41. Soufi, A., Garcia, M. F., Jaroszewicz, A., Osman, N., Pellegrini, M. and Zaret, K. S. (2015) Pioneer transcription factors target partial DNA motifs on nucleosomes to initiate reprogramming. Cell, 161, 555-568.
42. Shan, J., Fu, L., Balasubramanian, M. N., Anthony, T. and Kilberg, M. S. (2012) ATF4-dependent regulation of the JMJD3 gene during amino acid deprivation can be rescued in Atf4-deficient cells by inhibition of deacetylation. J Biol Chem, 287, 36393-36403.
43. Magnani, L., Ballantyne, E. B., Zhang, X. and Lupien, M. (2011) PBX1 genomic pioneer function drives ERa signaling underlying progression in breast cancer. PLoS Genet, 7, e1002368.
44. Wittkopp, P. J. and Kalay, G. (2011) Cis-regulatory elements: molecular mechanisms and evolutionary processes underlying divergence. Nat Rev Genet, 13, 59-69.
45. Zhao, Y., Sheng, H. Z., Amini, R., Grinberg, A., Lee, E., Huang, S., Taira, M. and Westphal, H. (1999) Control of hippocampal morphogenesis and neuronal differentiation by the LIM homeobox gene Lhx5. Science, 284, 1155-1158.
46. Lui, N. C., Tam, W. Y., Gao, C., Huang, J. D., Wang, C. C., Jiang, L., Yung, W. H. and Kwan, K. M. (2017) Lhx1/5 control dendritogenesis and spine morphogenesis of Purkinje cells via regulation of Espin. Nat Commun, 8, 15079.
47. Owens, G. K., Kumar, M. S. and Wamhoff, B. R. (2004) Molecular regulation of vascular smooth muscle cell differentiation in development and disease. Physiol Rev, 84, 767-801.
48. Stadler, M. B., Murr, R., Burger, L., Ivanek, R., Lienert, F., Schöler, A., van Nimwegen, E., Wirbelauer, C., Oakeley, E. J., Gaidatzis, D. et al. (2011) DNA-binding factors shape the mouse methylome at distal regulatory regions. Nature, 480, 490-495.
49. Domcke, S., Bardet, A. F., Adrian Ginno, P., Hartl, D., Burger, L. and Schubeler, D. (2015) Competition between DNA methylation and transcription factors determines binding of NRF1. Nature, 528, 575-579.
50. Karagiannis, P., Takahashi, K., Saito, M., Yoshida, Y., Okita, K., Watanabe, A., Inoue, H., Yamashita, J. K., Todani, M., Nakagawa, M. et al. (2019) Induced Pluripotent Stem Cells and Their Use in Human Models of Disease and Development. Physiol Rev, 99, 79-114.
51. Zenker, M., Bunt, J., Schanze, I., Schanze, D., Piper, M., Priolo, M., Gerkes, E. H., Gronostajski, R. M., Richards, L. J., Vogt, J. et al. (2019) Variants in nuclear factor I genes influence growth and development. Am J Med Genet C Semin Med Genet, 181, 611-626.
52. Qu, Y., Huang, Y., Feng, J., Alvarez-Bolado, G., Grove, E. A., Yang, Y., Tissir, F., Zhou, L. and Goffinet, A. M. (2014) Genetic evidence that Celsr3 and Celsr2, together with Fzd3, regulate forebrain wiring in a Vangl-independent manner. Proc Natl Acad Sci USA, 111, E2996-3004.
53. Laforest, B., Andelfinger, G. and Nemer, M. (2011) Loss of Gata5 in mice leads to bicuspid aortic valve. J Clin Invest, 121, 2876-2887.
54. Stankunas, K., Shang, C., Twu, K. Y., Kao, S. C., Jenkins, N. A., Copeland, N. G., Sanyal, M., Selleri, L., Cleary, M. L. and Chang, C. P. (2008) Pbx/Meis deficiencies demonstrate multigenetic origins of congenital heart disease. Circ Res, 103, 702-709.
55. Arrington, C. B., Dowse, B. R., Bleyl, S. B. and Bowles, N. E. (2012) Non-synonymous variants in pre-B cell leukemia homeobox (PBX) genes are associated with congenital heart defects. Eur J Med Genet, 55, 235-237.
56. Lambers, E., Arnone, B., Fatima, A., Qin, G., Wasserstrom, J. A. and Kume, T. (2016) Foxc1 Regulates Early Cardiomyogenesis and Functional Properties of Embryonic Stem Cell Derived Cardiomyocytes. Stem Cells, 34, 1487-1500.
57. Aston, C., Jiang, L. and Sokolov, B. P. (2005) Transcriptional profiling reveals evidence for signaling and oligodendroglial abnormalities in the temporal cortex from patients with major depressive disorder. Mol Psychiatry, 10, 309-322.
58. Waclaw, R. R., Allen, Z. J., 2nd, Bell, S. M., Erdelyi, F., Szabo, G., Potter, S. S. and Campbell, K. (2006) The zinc finger transcription factor Sp8 regulates the generation and diversity of olfactory bulb interneurons. Neuron, 49, 503-516.
59. Sarachana, T. and Hu, V. W. (2013) Genome-wide identification of transcriptional targets of RORA reveals direct regulation of multiple genes associated with autism spectrum disorder. Mol Autism, 4, 14.
60. Bhattaram, P., Penzo-Mendez, A., Kato, K., Bandyopadhyay, K., Gadi, A., Taketo, M. M. and Lefebvre, V. (2014) SOXC proteins amplify canonical WNT signaling to secure nonchondrocytic fates in skeletogenesis. J Cell Biol, 207, 657-671.
61. Mao, J., McGlinn, E., Huang, P., Tabin, C. J. and McMahon, A. P. (2009) Fgf-dependent Etv4/5 activity is required for posterior restriction of Sonic Hedgehog and promoting outgrowth of the vertebrate limb. Dev Cell, 16, 600-606.
62. Selleri, L., Depew, M. J., Jacobs, Y., Chanda, S. K., Tsang, K. Y., Cheah, K. S., Rubenstein, J. L., O'Gorman, S. and Cleary, M. L. (2001) Requirement for Pbxl in skeletal patterning and programming chondrocyte proliferation and differentiation. Development, 128, 3543-3557.
63. Koldobskiy, M. A., Jenkinson, G., Abante, J., Rodriguez DiBlasi, V. A., Zhou, W., Pujadas, E., Idrizi, A., Tryggvadottir, R., Callahan, C., Bonifant, C. L. et al. (2021) Converging genetic and epigenetic drivers of paediatric acute lymphoblastic leukaemia identified by an information-theoretic analysis. NatBiomedEng, 5, 360-376.
64. Gupta, S., Lafontaine, D. L., Vigneau, S., Vinogradova, S., Mendelevich, A., Igarashi, K. J., Bortvin, A., Alves-Pereira, C. F., Clement, K. and Pinello, L. (2020) DNA methylation is a key mechanism for maintaining monoallelic expression on autosomes. bioRxiv.
65. Brivanlou, A. H. and Darnell, J. E., Jr. (2002) Signal transduction and the control of gene expression. Science, 295, 813-818.
66. Martin-Trujillo, A., Patel, N., Richter, F., Jadhav, B., Garg, P., Morton, S. U., McKean, D. M., DePalma, S. R., Goldmuntz, E., Gruber, D. et al. (2020) Rare genetic variation at transcription factor binding sites modulates local DNA methylation profiles. PLoS Genet, 16, e1009189.
67. Plongthongkum, N., van Eijk, K. R., de Jong, S., Wang, T., Sul, J. H., Boks, M. P., Kahn, R. S., Fung, H. L., Ophoff, R. A. and Zhang, K. (2014) Characterization of genome-methylome interactions in 22 nuclear pedigrees. PLoS One, 9, e99313.
68. Gunasekara, C. J., Scott, C. A., Laritsky, E., Baker, M. S., MacKay, H., Duryea, J. D., Kessler, N. J., Hellenthal, G., Wood, A. C., Hodges, K. R. et al. (2019) A genomic atlas of systemic interindividual epigenetic variation in humans. Genome Biol, 20, 105.
69. Rizzardi, L. F., Hickey, P. F., Idrizi, A., Tryggvadóttir, R., Callahan, C. M., Stephens, K. E., Taverna, S. D., Zhang, H., Ramazanoglu, S., Hansen, K. D. et al. (2021) Human brain region-specific variably methylated regions are enriched for heritability of distinct neuropsychiatric traits. Genome Biol, 22, 116.
70. Churchill, G. A., Airey, D. C., Allayee, H., Angel, J. M., Attie, A. D., Beatty, J., Beavis, W. D., Belknap, J. K., Bennett, B., Berrettini, W. et al. (2004) The Collaborative Cross, a community resource for the genetic analysis of complex traits. Nat Genet, 36, 1133-1137.
71. Aylor, D. L., Valdar, W., Foulds-Mathes, W., Buus, R. J., Verdugo, R. A., Baric, R. S., Ferris, M. T., Frelinger, J. A., Heise, M., Frieman, M. B. et al. (2011) Genetic analysis of complex traits in the emerging Collaborative Cross. Genome Res, 21, 1213-1222.
72. Teschendorff, A. E. and Feinberg, A. P. (2021) Statistical mechanics meets single-cell biology. Nat Rev Genet.

TABLE 3
Comparison of methods for DNA methylation analysis¹

| | | | | | Amount of | | | |
|---|---|---|---|---|---|---|---|---|
| | Availability | | | | Starting | | | |
| Method | as a Kit | Coverage | Sensitivity | Specificity | Material | | ^{§} Price | References |
| Methods for Whole Genome Profiling | | | Whole Genome Assessment without Identification of | | | | | |
| | | | Differentially-Methylated Regions | | | | | |
| Mass spectrometry | No | Whole | *** detect | *** | 100 ng- | | $80/ | [18, 19, |
| (LC-MS/MS) | | genome | 5% | | 1 μg | | sample | 20, 21] |
| | | assessment | difference | | | | (Millis | |
| | | | in | | | | Scientific) | |
| | | | methylation | | | | | |
| | | | between | | | | | |
| | | | samples | | | | | |
| LUMA | No | Whole | ** | ** | 250-500 | ng | | [34, 38] |
| (Luminometric- | | genome | | | | | | |
| Based Assay) | | assessment | | | | | | |
| LINE-1 + | No | 17% of | *** | ** | 50 | ng | | [39] |
| pyrosequencing | | the whole | | | | | | |
| | | genome | | | | | | |
| HPLC-UV | No | Whole | * | ** | 3-10 | μg | | [17] |
| | | genome | | | | | | |
| | | assessment | | | | | | |

Methods for Identification of Differentially-Methylated Regions

| ^{#} Enrichment of | MethylCap | | *** | *** | 200 ng- | | $550/48 | [3, 40] |
|---|---|---|---|---|---|---|---|---|
| 5metC regions by | kit | | | | 1.2 μg | | rxns | |
| pulldown with | (Diagenode) | | | | | | | |
| MBD protein | MethylMiner | ~80% of | * | * | 5 ng- | | $485/25 | |
| (needs to be | Methylated | all 5-5 | | | 1 μg | | rxns | |
| followed by NGS | DNA | mCpG | | | | | | |
| or microarray) | Enrichment | | | | | | | |
| | Kit (Thermo | | | | | | | |
| | Fisher | | | | | | | |
| | Scientific) | | | | | | | |
| ^{#} Enrichment for 5 | MethylCollector | No info | ** | ** | 1 ng- | | $495/10 | [40, 41] |
| mC regions by | Ultra | | | | 1 μg | | rxns | |
| MBD2b/MBD3L1 | (Active | | | | | | | |
| proteins pulldown | Motif) | | | | | | | |
| (MIRA-based | | | | | | | | |
| assay) (needs to be | | | | | | | | |
| followed by NGS | | | | | | | | |
| or microarray) | | | | | | | | |
| ^{#} Enrichment for 5 | MeDIP | No info | No info | No info | 100 ng- | | $395/10 | [42, 43] |
| mC regions with | (Active | | | | 1 μg | | rxns | |
| antibodies (MeDIP) | Motif) | | | | | | | |
| (needs to be | MagMeDIP | No info | No info | No info | 1.2 | μg | $595/48 | [44] |
| followed by NGS) | (Diagenode) | | | | | | rxns; | |
| | | | | | | | $325/10 | |
| | | | | | | | rxns | |
| ^{#} Enrichment for | SureSelect | 3.7 × | *** | *** | 3 | μg | ~$320 | [5] |
| CpG rich regions | Human | 10⁶ CpGs | | | | | (baits); +$70 | |
| with RNA baits | Methyl-Seq | | | | | | (library | |
| (needs to be | (Agilent) | | | | | | prep) | |
| followed by NGS) | | | | | | | | |
| ^{#} Enrichment for | SeqCap Epi | 5.5 × | *** | *** | 1 | μg | ~$450; +bisulfite | [45, 46] |
| CpG rich regions | CpGiant | 10⁶ CpGs | | | | | conversion | |
| by hybridisation | Enrichment | | | | | | kit | |
| with bait | Kit (Roche) | | | | | | | |
| oligonucleotides | | | | | | | | |
| (needs to be | | | | | | | | |
| followed by NGS) | | | | | | | | |
| HumanMethylation | Illumina | 482.000 | *** | *** | 0.5-1 | μg | ~$9000/ | [47] |
| 450 BeadChip array | | CpG sites | | | | | 24 | |
| | | (99% of | | | | | samples | |
| | | known | | | | | (2 chips) | |
| | | genes) | | | | | | |
| Sequencing of | Illumina | varies, | varies, as | Depends | >50 | ng | $360/ | |
| methylation- | | depending | number of | on the | | | sample | |
| enriched fraction of | | on the | reads | enrichment | | | | |
| the genome (after | | sample | correlates | method | | | | |
| MeDIP or MIRA) | | | with the | | | | | |
| | | | amount of | | | | | |
| | | | DNA | | | | | |
| Whole genome | Illumina | 100% | | *** | 50-100 | ng | $6000/ | [48, 49] |
| bisulfite sequencing | | | | | | | sample | |
| (WGBS) | | | | | | | | |
| Reduced | Illumina | ~60% of | varies, as | *** | 1 | μg | $2700- | [50, 51] |
| representation | | promoters, | sequencing | | | | 5000/ | |
| bisulfite sequencing | | 85% of | depth | | | | sample | |
| (RRBS) | | CGI (~1.5 × | correlates | | | | (sequencing | |
| | | 10⁶ CpGs) | with the | | | | and | |
| | | | amount of | | | | library | |
| | | | DNA (20X | | | | prep) | |
| | | | coverage is | | | | | |
| | | | recommended) | | | | | |
| Methyl-MiniSeq | Zymoresearch | >85% | varies, as | | 100 ng- | | $2800 | [44, 52] |
| (improved RRBS) | | coverage | sequencing | | 5 μg | | (all steps | |
| | | of all CpG | depth | | | | and | |
| | | islands | correlates | | | | bioinformatics | |
| | | and >80% | with the | | | | included | |
| | | of all gene | amount of | | | | | |
| | | promoters | DNA (20X | | | | | |
| | | (~3 × | coverage is | | | | | |
| | | 10⁶ CpGs) | recommended) | | | | | |
| Methyl-sensitive | No | 1% of all | depends on | *** | 1-5 | μg | | [53] |
| cut counting | | CpG | the | | | | | |
| (MSCC) (needs to | | | sequencing | | | | | |
| be followed by | | | coverage | | | | | |
| NGS) | | | | | | | | |
| Improved MSCC | No | 30% of | | *** | 1-5 | μg | | [54, 55] |
| (needs to be | | CpGs | | | | | | |
| followed by NGS) | | (~58% of | | | | | | |
| | | CpG-rich | | | | | | |
| | | regions) | | | | | | |

Low Throughput Methods

| | | | | | | | |
|---|---|---|---|---|---|---|---|
| PCR-based | OneStep | Gene- | *** | *** | ≥20 | ng | [56] |
| (digestion | qMethyl Kit | specific | | | | | |
| followed | (Zymoresearch) | 22 | | | | | |
| by PCR) | EpiTect II | samples/ | | | 1-4 | μg | [57] |
| | DNA | 96-well | | | | | |
| | methylation | plate | | | | | |
| | enzyme Kit | | | | | | |
| | (Qiagen) | | | | | | |

^{#} Enriched fractions are normally used for NGS.
^{§} All prices are approximate and have been obtained during 2015.
*** Defines the best sensitivity or specificity (*** > ** > *).
¹Sergey Kurdyukov and Martyn Bullock, Biology (Basel). 2016 March; 5(1): 3, 2016 “DNA Methylation Analysis: Choosing the Right Method”

Although the invention has been described with reference to the above examples, it will be understood that modifications and variations are encompassed within the spirit and scope of the invention. Accordingly, the invention is limited only by the following claims.

1. A method of detecting a developmental defect in a subject comprising identifying changes in a normalized methylation entropy (NME) level in a sample from the subject, thereby detecting the developmental defect in the subject.

2. A method of detecting cancer, cancer recurrence and/or cancer recurrence risk in a subject comprising identifying changes in a normalized methylation entropy (NME) level in a sample from the subject, thereby detecting cancer, cancer recurrence and/or cancer recurrence risk in the subject.

3. The method of claim 1, wherein identifying changes in an NME level comprises identifying a change in DNA methylation landscape in the sample from the subject.

4. The method of claim 3, wherein identifying a change in methylation landscape comprises calculating an information-theoretic distance between a methylation potential energy landscape quantified by an uncertainty coefficient (UC) in the sample from the subject and a methylation potential energy landscape quantified by an UC in a reference sample.

5. The method of claim 4, wherein the UC measures an inherent information in a sample corrected for entropy.

6. The method of claim 4, further comprising deriving tissue-specific developmental trajectories from the information-theoretic content of DNA methylation, wherein identifying regions with high UC in the sample from the subject as compared to the UC in the reference sample indicates a developmental change in the subject.

7. The method of claim 6, wherein the developmental change is a tissue-specific developmental change or a time-dependent developmental change.

8. The method of claim 7, wherein the tissue-specific developmental change comprises changes in embryonic facial prominence, liver, heart, limb, forebrain, midbrain and/or hindbrain developmental change.

9. The method of claim 6, wherein the developmental change comprises epithelial to mesenchymal transition.

10. The method of claim 6, wherein deriving tissue-specific developmental trajectories comprises identifying entropy-associated motifs undergoing methylation landscape changes.

11. The method of claim 10, wherein the entropy-associated motifs are selected from the motifs identified in Table 2.

12. The method of claim 1, wherein changes in the normalized methylation entropy (NME) level are determined for each allele.

13. The method of claim 2, wherein identifying changes in an NME level comprises identifying a change in DNA methylation landscape in the sample from the subject.

14. The method of claim 2, wherein changes in the normalized methylation entropy (NME) level are determined for each allele.
